(19) United States
(12) Patent Application Publication
(10) Pub. No.: US 2018/0120294 A1
(43) Pub. Date: May 3, 2018

(54) Microfluidic Devices for Cells or Organ based Multimodal Activation and Monitoring system

(71) Applicant: JOHN COLLINS, Irvine, CA (US)

(72) Inventor: JOHN COLLINS, Irvine, CA (US)

(21) Appl. No.: 15/339,379

(22) Filed: Oct. 31, 2016

(51) Int. Cl. G01N 33/50; B01L 3/00

(52) U.S. Cl. CPC G01N 33/502; G01N 33/5058; G01N 33/5061; B01L 2300/0645; B01L 2300/0887; B01L 2300/0816; B01L 3/502715

(57) ABSTRACT

Chip, system and method using microfluidic devices and instrumentation for multiple parameter stimulation and recording on cells or organs is presented. The chip consists of multilayer fluidic channels to organize cells or organs and to flow stimulant molecules or cells. Further an array of such channels are arranged spatially for multiple biochemical reaction or assay or co-culture. The reactors are also arranged in one or more spiral fluidic channel with multiple connecting channels between two or more such spiral channels. The system consists of stimulation instrumentation for the cells or organs using optical, electrical, mechanical, fluidic and chemical and recording instrumentation for signals or images from the cells simultaneously or alternatively are performed using optical imaging, electrical field potential and impedance. Such system is operated remotely from an incubator or microscopic sterile environment using wireless or wired networks. The methodology for performing assay and drug screening utilizing several functional activation and measurement parameters is presented.

### CROSS-REFERENCE TO RELATED APPLICATIONS

The present application claims the benefit of and priority to U.S. Provisional Patent Application titled “Microfluidic Devices for Cells or Organ based Multimodal Activation and Monitoring system,” Ser. No. 62/249,271, filed on Nov. 1, 2015. The disclosure in this provisional application is hereby incorporated fully by reference into the present application.

### STATEMENT REGARDING FEDERALLY-SPONSORED RESEARCH AND DEVELOPMENT

This invention was made with Government support under contract No. R43HL118938 and R43MH104170 awarded by the National Institute of Health (NIH). The Government has certain rights in this invention.

### FIELD OF THE INVENTION

The present invention generally relates to medical devices and methods, and more particularly to microfluidic devices and methods for multiplexed cell based assays and multimodal activation and monitoring systems.

### BACKGROUND OF THE INVENTION

Microfluidic systems provide remarkable features for controlling the fluidics in cell based assays. Fluidic circuits mix two or more reagents, develop multiple composition of reagents, perform concentration gradient and periodically deliver fluids. Monitoring systems probe cellular systems for growth or signaling due to activation parameters not limited to optical, electrical, mechanical, chemical and acoustics.

### SUMMARY

The present invention is directed to a system and method for cell based assays using microfluidic system equipped with perfusion, stimulation using optical, chemical, mechanical, acoustics and electrical and monitoring using optical imaging, electrical field potentials, electrical impedance, fluidic pressure, substantially as shown in and/or described in connection with at least one of the figures, and as set forth more completely in the claims.

In accordance with an aspect of the present invention, there are provided methods for performing high-throughput cell based assay using microfluidic reactors designed in standard formats such as 96 well-plate format and customized well or reactors format.

In accordance with another aspect of the present invention, there are provided methods for performing concentration gradient using splitting fluidic flow from two or more inputs of drug, growth factor, toxin, stimuli agents, other chemicals or reagents.

In accordance with yet another aspect of the present invention, there are provided methods for performing fluidic flow from one reactor not entering to another reactor.

In accordance with yet another aspect of the present invention, there are provided methods for collecting the cells in inner reactor within a reactor with cells and reagents trapped inside.

In accordance with yet another aspect of the present invention, there are provided methods for collecting used reagents from one well in to a common outlet using binary split channels.

In accordance with yet another aspect of the present invention, there are provided methods for transporting fluids to or from individual reactors using larger channels which offer low fluidic resistance to transport equally in to the reactors.

In accordance with yet another aspect of the present invention, there are provided methods for forming serial high-throughput reactors using primary and secondary spiral channel where the inlets are connected to primary channel and outlets are connected to secondary channel.

In accordance with yet another aspect of the present invention, there are provided methods for carrying out concentration gradient on the serial reactors in pair of spiral channels by ramping the flow rates of two or more fluids in an increasing flow rate for one fluids and decreasing flow rate for the second fluids or vice versa or combination of linear or non-linear order.

In accordance with yet another aspect of the present invention, there are provided methods for the monitoring biochemical reaction or cell growth or cell differentiation or cell phenotype change in the reactors using optical sensors, or impedance or field potential sensors using electrodes form on the surface as a two-dimensional (2-d) or three-dimensional (3-d) formats.

In accordance with yet another aspect of the present invention, there are provided methods for performing impedance or field potential measurements or electrical stimulation or both using electrodes on glass or printed circuit board or plastics substrate.

In accordance with yet another aspect of the present invention, there are provided methods for performing high-throughput screening using open well or closed reactor format and feeding fluids in the reactors and removing fluids from the reactors.

In accordance with yet another aspect of the present invention, there are provided methods for fabrication of the fluidic-electric chip using multilayer chips using injection molded parts, printed circuited parts, multilayer-bonded parts or micromachined parts with one or more top cover to successively close the fluidic ports for biochemical processing.

In accordance with yet another aspect of the present invention, there are provided electrode and fluidic chips with fluidic ports on the top of the chip or manifold and electrical pads in the bottom of the chip or manifold or vice versa.

In accordance with yet another aspect of the present invention, there are provided methods for performing cell culture on a chip using one set of ports for cell entry and another set of ports for perfusion fluidic entry.

In accordance with yet another aspect of the present invention, there are provided methods for performing coculture of two or more cells such as neural cells, cardiac cells, muscle cells or any other cells within a fluidic chip using two or more layers of fluidics separated by one or more filters of sizes depending on the cells used diffusion or convection of fluids within the system.

In accordance with yet another aspect of the present invention, there are provided methods for monitoring cells within a co-culture system using impedance or field potential measurements.

In accordance with yet another aspect of the present invention, there are provided methods for screening drugs or toxins or differentiation of pluripotent stem cells using two or more cell types within two or more compartments of fluidics separated by filters

In accordance with yet another aspect of the present invention, there are provided methods for performing coculture of cells in channels with ellipsoidal shaper or spiral shape or other fluidics favorable shaped channels.

In accordance with yet another aspect of the present invention, there are provided methods for separation of a cell population from mixture of cells using successive size based separation in spiral fluidic channels separated by filters of increasing sizes using a block of sandwiched spirals and filter.

In accordance with yet another aspect of the present invention, there are provided methods for performing assays in series of reactors fitted with impedance or field potential monitoring electrodes and transparent windows for optical monitoring.

In accordance with yet another aspect of the present invention, there are provided methods for lateral flow of cells or fluids in spiral channels with cell or molecules separation through filters throughout the cross section of the spiral channels and transporting the collected fluids or cells to another set of channels through a separating layer of connecting channels.

In accordance with yet another aspect of the present invention, there are provided methods for mechano stimulation of cells using fluidic shearing through pumps integrated on chip or through external pumps.

In accordance with yet another aspect of the present invention, there are provided methods for pumping fluids using one or more single or pairs of piezo electric transducers and actuators (PZT) or PZT blenders using alternating current (AC) voltages in cascaded or perstastic modes of operation with tesla, diffuser/nozzle valve or combinations separating different stages to direct the pumping.

In accordance with yet another aspect of the present invention, there are provided methods for actuating the PZTs using trapezoidal wave form AC voltage signals so that the PZT rapidly push the fluid to the next stage reservoir and slowly fill its own reservoir.

In accordance with yet another aspect of the present invention, there are provided methods for fabrication of co-culture cell chip to perform endothelial tight junction based cell assay using multiple layers of channels, two set of inlets, and two sets of outlets in each compartment with all the inlets and outlets originating from the top most layer to connect to a manifold.

In accordance with yet another aspect of the present invention, there are provided methods to equip multiple electrodes for monitoring cell growth or cell behavior using electrical impedance measurements and/or field potential measurements within each compartment or between the compartments.

In accordance with yet another aspect of the present invention, there are provided methods for separating cells from each compartment using filters and to overlap channels to expose the filter in certain area of filter in the form of elliptical or circular or square ring of a specific width and diameter to contain the chambers.

In accordance with yet another aspect of the present invention, there are provided methods for viewing or imaging cells in a specific area of the reactor or compartments while the compartments are arranged in concentric ellipses or circles or squares so that both the compartments can be viewed together.

In accordance with yet another aspect of the present invention, there are provided methods for pumping and filling fluids or priming in a chamber using three step operation by priming the side finger channels before cells input, filling the chamber with cells through main inlet and to continue perfusion through the side fingers.

In accordance with yet another aspect of the present invention, there are provided methods for configuring electrodes for stimulation by choosing an electrode with cells or a middle electrodes and monitoring cells using an array of electrodes configured in the flow path of an elliptical or circular chamber or channel and monitoring cells using interdigitated electrodes or fractal electrodes.

In accordance with yet another aspect of the present invention, there are provided methods for configuring ground or reference electrodes in the middle of the four quadrants of the chamber as a cross or long electrode on a side of the chamber.

In accordance with yet another aspect of the present invention, there are provided methods for configuring channels in a 1-d format either as single ended electrodes with reference or ground electrodes on a side or as a single ended electrode with vias on every electrodes so that ground or reference electrodes can be around them or as differential electrodes.

In accordance with yet another aspect of the present invention, there are provided methods for configuring channels in a 1-d format either as single ended electrodes with reference or ground electrodes on a side or as a single ended electrode with vias on every electrodes so that ground or reference electrodes can be around them or as differential electrodes.

In accordance with yet another aspect of the present invention, there are provided methods for generating electrical stimulus signals with biphasic pulse train or monophasic with positive or negative pulses with interphase delays with a phase difference between the pulses shaped as sine wave, triangle wave, square wave, trapezoidal wave or an arbitrary custom drawn signal waveform.

In accordance with yet another aspect of the present invention, there are provided methods for generating optogenetic stimulation using light of a particular wavelength and magnitude with a fixed period or wave form train shaped as sine wave, triangle wave, square wave, trapezoidal wave or an arbitrary custom drawn signal waveform or combinations of optical stimulus pulse coupled with or without electrical stimulation.

In accordance with yet another aspect of the present invention, there are provided methods for stimulating the cells using optogenetic light of a particular wavelength and magnitude with a fixed period or wave form train shaped as sine wave, triangle wave, square wave, trapezoidal wave or an arbitrary custom drawn signal waveform or combinations of optical stimulus pulse coupled with or without electrical stimulation while monitoring field potential signals or impedance signals or intermittent optical signals.

In accordance with yet another aspect of the present invention, there are provided methods for stimulating cells using chemicals or combinations of chemicals for a fixed period of fluidic pulses or constant perfusion flow using a specific concentration or gradient of concentrations or composition of chemicals across chambers.

In accordance with yet another aspect of the present invention, there are provided methods for stimulating cells using chemicals or combinations of chemicals across multiple concentric spiral micro/nano spaced interconnected channels where multiple inlets and outlets of the spiral channels serve as inlets and outlets to stimulant or cells forming tight junctions to transport fluids across multiple channels for drug screening application assessed by trans-epithelial electrical resistance measurements.

In accordance with yet another aspect of the present invention, there are provided methods for building a manifold using serpentine or ellipse channel or cylindrical reservoirs and pumps monitoring by pressure sensor to release fluidic pressure using a set of valves.

In accordance with yet another aspect of the present invention, there are provided methods for building a multilayer manifold for securing the chip fluidics using sealing gaskets and electrodes using spring loaded connector fixture and strong magnet or pressure control require the hold the manifold together.

In accordance with yet another aspect of the present invention, there are provided methods for containing different reagents to feed the cells, stimulate the cells or monitor the cells using chemical reagents and for releasing the fluids in different containers using pressure controlled valves and pumps.

In accordance with yet another aspect of the present invention, there are provided methods for securing manifold and chips and electronic instrumentation in a compact form and to perform further biochemical analysis such as PCR or immunoassay or oxidation reduction reaction for analysis of products after screening assay.

In accordance with yet another aspect of the present invention, there are provided methods for performing electrochemical measurements using two, three or more electrodes in the reactors and to perform non-faradaic high speed impedance measurement to correlate with other monitoring systems.

In accordance with yet another aspect of the present invention, there are provided methods for sensing the cells using differential impedance measurement of neighboring electrodes from the top, bottom, right or left to stimulate the cells or to measure field potential signals.

In accordance with yet another aspect of the present invention, there are provided methods for measuring voltages from the cells or stimulating the cells using current or voltage pulses using a Field Point Gated Array (FPGA) or microcontroller equipped with memories including flash, SDRAM, SRAM and hard drive and to control the devices such as voltage pulse generator, fluidic pumps and valves and memory transaction as well as transmit data from the voltage amplifiers/Data acquisition (DAQ) system to a remote server wirelessly or through wire.

In accordance with yet another aspect of the present invention, there are provided methods for developing double sided printed circuit board (PCB) with electrical circuits with electromagnetic shielding of low voltage signals on one side and pads of bio fluidic chips on another side.

In accordance with yet another aspect of the present invention, there are provided methods for operating the pumps, valves, timing electronics of pulsed fluidics or electrics and to transmit data or images from the chip to remote sensor using a portable system operating on a battery or ac electric power.

In accordance with yet another aspect of the present invention, there are provided methods for programming pumps, valves and pressure sensor to control fluidics on the biochip using FPGA or microcontroller or computer programs either as a simple open loop system or as a closed loop system using sensors for impedance from the cells, pressure from the reservoir, fluid level from the reservoir and cell growth control.

In accordance with yet another aspect of the present invention, there are provided methods for building a high-throughput field potential or impedance measurement system from multiple electrodes using an array of cascaded amplifiers or circuits and stimulation system using an array of voltage generator circuits.

In accordance with yet another aspect of the present invention, there are provided methods for displaying data on a screening for user view and analysis and control of devices and storage or transmission of data.

In accordance with yet another aspect of the present invention, there are provided methods for securing, communicating electrical signals and recording optical images from biochip using multiple layers of PCB fixture, fluidic manifold with opening at the middle to optical transparency.

In accordance with yet another aspect of the present invention, there are provided methods for drug study and analysis using field potential signal spikes sorted through spike sorting algorithms for uniformity of the cells under study and performing statistical analysis with uniformity and normalized data to establish the effect of drug on the cells under study.

In accordance with yet another aspect of the present invention, there are provided methods for analysis of drug screening includes spikes characteristics using valley width and amplitude, peak width and amplitude, ratio of peak and valley characteristics, spike rate and burse rate or combination of above said parameters.

In accordance with yet another aspect of the present invention, there are provided methods for integrating the system for users using an environmental controlled computer operated system with an optical microscope and electrical measurements or a remote-controlled system to operate within a specific third party multimodal measurement system or independently operated system located in a general-purpose incubator.

In accordance with yet another aspect of the present invention, there are provided methods for portable system with multiple measurement and stimulation for measurement of optical imaging while measuring field potential or impedance data from the cells under perfusion of media or drug and stimulations including optical stimulation, mechanical stimulation and electrical stimulation.

In accordance with yet another aspect of the present invention, there are provided methods for data communication across bioreactor experimental system on demand from a remote terminal and integration of multimodal measurements such as optical images, gene expression data and patient's in vivo measurements such as electrocardiogram (ECG) or electrocorticography (ECoG) or optogenetic signals to transmit between investigators and clinicians through Biopico Systems' support team.

In accordance with yet another aspect of the present invention, there are provided methods for operating fluidics chip together with fluidic manifold, electrical instrumentation, software control and statistical analysis to perform toxicological screening, pharmacological screening, disease modeling and personalized medicine study using pluripotent stem cells or differentiated cells.

In accordance with yet another aspect of the present invention, there are provided methods for personalized medicine through skin biopsy from patients and screening the derived induced pluripotent cells under differentiation for drug discovery or optimization with multiple drug cocktails for patient specific pharmacology of diseases.

In accordance with yet another aspect of the present invention, there are provided methods for performing biochemical analysis such as polymerase chain reaction (PCR), immunohistochemsitry, flow cytometry from the recovered cells from reactors after performing field potential or impedance monitoring experiment under perfusion of media or drug or toxins to validate, reinforce and complement toxicology or pharmacology study.

In accordance with yet another aspect of the present invention, there are provided protocol for performing drug screening experiments with specific times of exposure to consecutive concentrations from low to high value during the field potential, impedance or optical monitoring of diseased cells studied with control cells for optimization and normalization.

In accordance with yet another aspect of the present invention, there are provided methods for cell based assay include stimulation of the cells using fluidic shear for mechano stimulation, optical stimulation, chemical stimulation or combination and to establish the assay using multimodality measurements such as electrical, optical, pressure measurements for clinical evaluations.

Further aspects, elements and details of the present invention are described in the detailed description and examples set forth here below.

### BRIEF DESCRIPTION OF THE DRAWINGS

The embodiments set forth in the drawings are illustrative and exemplary in nature and not intended to limit the subject mater degined by the claims. The following detailed description of the illustrative embodiments can be understood when read in conjunction with the following drawings, where like structure may be indicated with like referene numberals in which:

FIG. 1A shows a diagram of an exemplary microfluidic multimodal activation and monitoring system for cells or organ;

FIG. 1B shows a diagram of an exemplary high-throughput screening chip;

FIG. 1C shows a diagram of an exemplary high-throughput screening chip with separate perfusion channels in another layer of the chip and a filter layer for securing the cells from perfusion channel flow;

FIG. 2 shows an high-throughput screening chip with larger channels at inlets or outlets and electrode arrays at the bottom of the reactors to record impedance or field potential signals;

FIG. 3 shows serial high-throughput reactors using primary and secondary spiral channel where the inlets are connected to primary channel and outlets are connected to secondary channel;

FIG. 4A shows a microelectrode array with electrodes for stimulation, monitoring and ground for two wells;

FIG. 4B shows a microelectrode array for high-throughput Chip in 96-well format;

FIG. 4C shows high-throughput fluidic chip using open well or closed reactor format;

FIG. 5A shows a diagram of exemplary design details of a fluidic layer and an electrodes layer;

FIG. 5B shows transparent fluidic channel and gold electrodes on printed circuit board and connection pads at the bottom;

FIG. 6A shows schematics of two inlets & 2 outlets perfusion chip;

FIG. 6B shows coculture fluidic chip using layers of fluidics with inlet and outlet channels separated by filters;

FIG. 7 shows a co-culture disease modeling chip using microfluidics system using two or more cell types within two or more compartments of fluidics separated by filters;

FIG. 8 shows coculture of cells in channels with spiral shape fluidic channels;

FIG. 9 shows separation of a cell population from mixture of cells using successive size based separation in spiral fluidic channels separated by filters of increasing sizes;

FIG. 10 shows mechano stimulation of cells using fluidic shearing through pumps integrated on chip piezo electric actuators (PZT) pumps;

FIG. 11A shows pumping fluids using pairs of PZT blenders in cascaded with combinations of tesla and diffuser/nozzle valves for separating different stages to direct the pumping;

FIG. 11B shows actuating the PZTs using trapezoidal wave form ac voltage signals so that the PZT rapidly push the fluid to the next stage reservoir and slowly fill its own reservoir;

FIG. 12A shows methods for fabrication of co-culture cell chip using multiple layers of channels two set of inlets and two sets of outlets in each compartment;

FIG. 12B shows the side view of all the inlets and outlets originate from the top most layer to connect to a manifold;

FIG. 12C shows the top view of one of the top layers to flow endothelial cells to perform tight junction endothelial layer;

FIG. 12D shows the top view of one of the top layers to flow perfusion of media to perform tight junction endothelial layer;

FIG. 12E shows the top view of one of the bottom layers to perform cell based assay within tight junction;

FIG. 12F shows the top view of one of the bottom layers to perfuse to perform cell based assay within tight junction;

FIG. 13A shows the top view of top most electrode layer to measure tight junction impedance at multiple positions;

FIG. 13B shows the top view of one of the top layers with a provision to view the cells;

FIG. 13C shows the top view of one of the top layers with a provision to bridge the fluidics from another top layer and perfusion;

FIG. 13D shows the top view of one of the bottom layers with an overlap to expose filter layer that forms a substrate for building tight junction with endothelial layer;

FIG. 13E shows the top view of one of the bottom layers with a provision to perform perfusion;

FIG. 13F shows the top view of bottom most electrode layer to measure tight junction impedance from the top most layer at multiple positions and to perform field potential measurements for cell based assay;

FIG. 14A shows a Computational Fluid Dynamics (CFD) based design of operation step for priming from side channels;

FIG. 14B shows a CFD based design of operation step for cells flow from main channel;

FIG. 14C shows a CFD based design of operation step for cell perfusion from side channels;

FIG. 14D shows high-throughput cell based assay using the CFD design and operation so that cells from one reactor will not enter another reactor;

FIG. 15A shows configuring electrodes for stimulation by choosing an electrode with cells or a middle electrode;

FIG. 15B shows configuring electrodes for monitoring cells using an array of electrodes configured in the flow path of an elliptical chamber;

FIG. 15C shows configuring electrodes monitoring cells using fractal electrodes;

FIG. 15D shows configuring electrodes monitoring cells using interdigitated electrodes;

FIG. 16A shows a 60-electrode array chip with pads on the top and bottom of the chip for connecting to amplifiers;

FIG. 16B shows configuring ground or reference electrodes in the middle of the four quadrants of the chamber as a cross;

FIG. 16C shows configuring ground or reference electrodes in the middle of the four quadrants of the chamber as long electrode on a side of the chamber;

FIG. 17A shows configuring electrodes in channel in a 1-d format as single ended electrodes with reference or ground electrodes on a side;

FIG. 17B shows configuring electrodes in channel in a 1-d format as single ended electrodes with reference or ground electrodes as single ended electrodes with vias on every electrode so that ground or reference electrodes are around them;

FIG. 18 shows configuring electrodes in channel in a 1-d format as single ended electrodes with reference or ground electrodes as differential electrodes;

FIG. 19 shows generating optogenetic stimulation using light of a particular wavelength and magnitude coupled with electrical stimulation and recording;

FIG. 20A shows stimulating cells using chemicals or combinations of chemicals using a specific concentration or gradient of concentrations or composition of chemicals across reactors;

FIG. 20B shows stimulating cells using chemicals or combinations of chemicals across multiple concentric spiral micro/nano spaced interconnected channels;

FIG. 21A shows for building a manifold using serpentine or ellipsoidal channels and pump;

FIG. 21B shows for building a manifold using cylindrical reservoirs monitoring by pressure sensors to release fluidic pressure using a set of valves;

FIG. 22 shows building a multilayer manifold for securing the chip fluidics using sealing gaskets and electrodes using spring loaded connector fixture and strong magnet to hold the manifold together;

FIG. 23 shows containers for different reagents to feed the cells, stimulate the cells or monitor the cells using chemical reagents and for releasing the fluids in different containers using pressure controlled valves and pumps;

FIG. 24A shows securing manifold and chips and electronic instrumentation in a compact form;

FIG. 24B shows biochemical analysis such as PCR or immunoassay for analysis of products after screening assay;

FIG. 25 shows electrochemical measurements using three electrodes in the reactors and to perform oxidation reduction reaction to correlate with other monitoring systems;

FIG. 26 shows non-faradaic high speed impedance measurement for monitoring cell activity;

FIG. 27A shows sensing the cells using differential impedance measurement of neighboring electrodes to stimulate the cells or to measure field potential signals;

FIG. 27B shows measurement of differential impedance from the top, bottom, right or left of a sensing electrode;

FIG. 28 shows measuring voltages from the cells or stimulating the cells using current or voltage pulses using a FPGA and transmit data from the voltage amplifiers/DAQ system to a remote server wirelessly;

FIG. 29 shows developing double sided PCB with electrical circuits with electromagnetic shielding of low voltage signals on one side and pads of bio fluidic chips on another side;

FIG. 30 shows operating the pumps, valves, timing electronics of pulsed fluidics or electrics and to transmit data or images from the chip to remote sensor using a portable system operating on a battery;

FIG. 31A shows pumps, valves and pressure sensor to control fluidics on the biochip;

FIG. 31B shows programming and control of fluidics to release fluid in to biochip;

FIG. 32A shows a simple open loop system to operate priming, cells loading and perfusion;

FIG. 32B shows a closed open loop system using sensors for impedance from the cells to operate priming, cells loading and perfusion;

FIG. 32C shows a closed open loop system using sensors for impedance from the cells to operate priming, cells loading and perfusion as a portable wireless system;

FIG. 32D shows a closed open loop system using sensors for impedance from the cells to operate priming, cells loading and perfusion as a portable wireless system with fluidic level monitoring from the reservoir;

FIG. 33 shows building a high-throughput field potential or impedance measurement system from multiple electrodes using an array of cascaded amplifiers or circuits and stimulation system using an array of voltage generator circuits;

FIG. 34A shows displaying data on a screening assay for user view and analysis and control of devices and storage or transmission of data;

FIG. 34B shows flow chart of operation of a screening assay for analysis, control of devices and storage or transmission of data;

FIG. 35 shows mechanical system for securing, communicating electrical signals and recording optical images from biochip using multiple layers of PCB fixture, fluidic manifold with opening at the middle to optical transparency;

FIG. 36 shows methods for drug study and analysis using field potential signal spikes sorted through spike sorting algorithms for uniformity of the cells under study and performing statistical analysis with uniformity and normalized data to establish the effect of drug on the cells under study;

FIG. 37 shows analysis of drug screening includes spikes characteristics using valley width and amplitude, peak width and amplitude, ratio of peak and valley characteristics;

FIG. 38 shows integrating portable system with multiple measurements and stimulations spatially located on top, bottom or side of the chip;

FIG. 39 shows integrating the cell based assay system using an environmental controlled computer operated system with an optical microscope and electrical measurements;

FIG. 40 shows integrating the system for portable and remote controlled operation;

FIG. 41A shows integrating the system to operate within a specific third party multimodal measurement system;

FIG. 41B shows measurement of temporal multimodal measurement remotely;

FIG. 42 shows integrating as an independently operable system located in a general-purpose incubator;

FIG. 43 shows system with disposable to chip placed on a tray in the device for automatic and user friendly operation;

FIG. 44 shows integrating optical imaging with the electrical measurement system in a remote-controlled system;

FIG. 45 shows personalized medicine through skin biopsy from patients and screening the derived induced pluripotent cells under differentiation for patient specific drug discovery of diseases;

FIG. 46 shows flow chart for operating fluidics chip together with fluidic manifold, electrical instrumentation, software control and statistical analysis to perform toxicological screening;

FIG. 47 shows flow chart for performing biochemical analysis such as PCR, from the recovered cells from reactors;

FIG. 48 shows protocol for performing drug screening experiments with control cells for optimization and normalization;

FIG. 49 shows flow chart for cell based assay include stimulation of the cells using fluidic shear for mechano stimulation and multimodality measurements;

FIG. 50A shows data communication across bioreactor experimental system on demand from a remote terminal and integration of multimodal optical measurements;

FIG. 50B shows data communication across bioreactor experimental system on demand from a remote terminal and integration of multimodal gene expression data; and

FIG. 50C shows data communication across bioreactor experimental system on demand from a remote terminal and integration of multimodal ECG signals to transmit between investigators and clinicians through Biopico Systems' support team.

### DETAILED DESCRIPTION

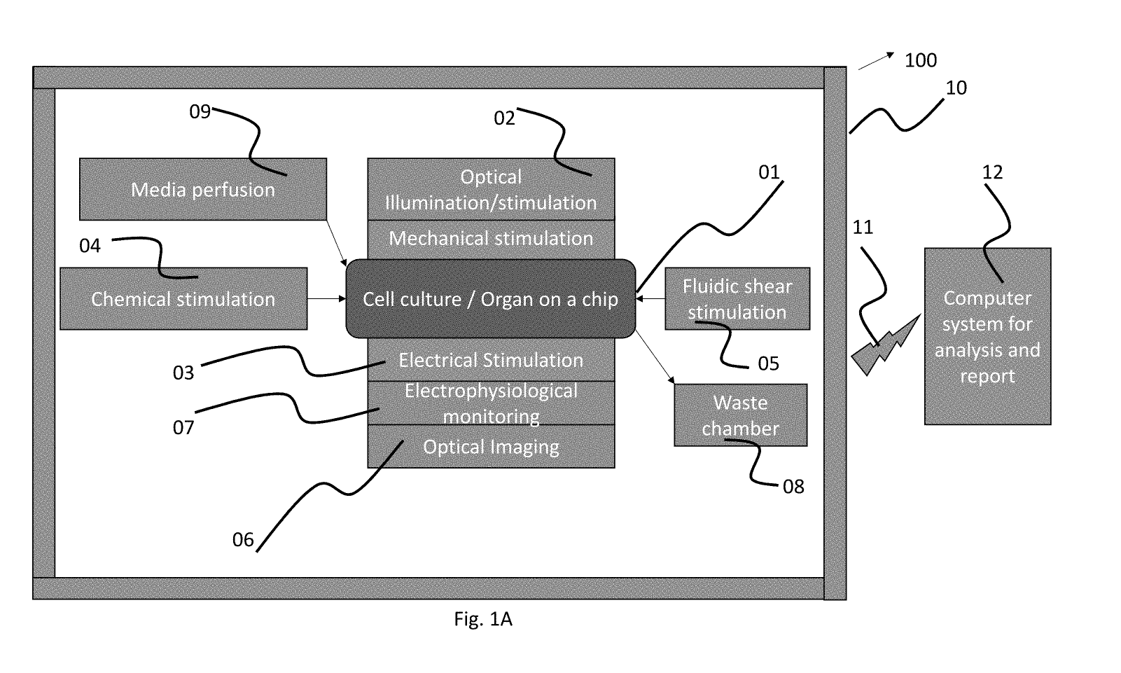

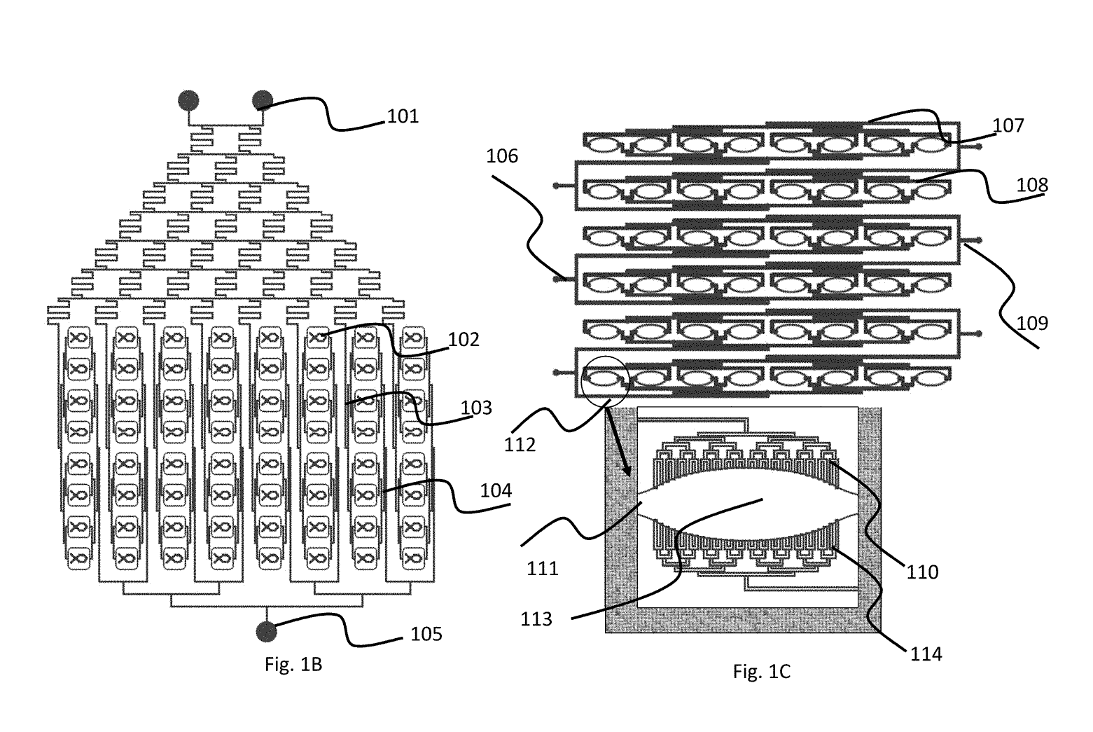

The following description contains specific information pertaining to implementations in the present application. The drawings in the present application and their accompanying detailed description are directed to merely exemplary implementations. Unless noted otherwise, like or corresponding elements among the figures may be indicated by like or corresponding reference numerals. Moreover, the drawings and illustrations in the present application are generally not to scale, and are not intended to correspond to actual relative dimensions. FIG. 1A shows system 100 including multilayer chip 01. System 100 may include multimodal stimulation, such as optical illumination/stimulation 02, electrical stimulation 03, chemical stimulation 04, and fluidic shear stimulation 05. System 01 may include devices for recording multiple parameters, such as optical imaging 06, and electrophysiological monitoring 07 enhanced with media or reagent perfusion fluidics 09. The system may be adapted in lab incubator 10. Data from the system to record signals and to control to the system may be carried out remotely using a wired network or wireless network connected to computer system 12 via connection 11. The data will be further processed for any pharmaceutical, clinical or research report

### Design and Development of Chip

High Throughput Chip:

A high-throughput chip is disclosed having a plurality of layers. In some implementations, the high-throughput chip may include one or more layers of fluidic channels to offer high-throughput reactors in a 2-d array forming a foot print of a standard well-plate with gradient generator as shown in FIG. 1B. Cells may be loaded at the inlet 101. There are provisions for collecting the cells in inner cup 102 or within a reactor having cells and reagents trapped inside. In some implementations, fluid entering inlet 103 of a first reactor will not enter another reactor. Instead, the fluid is directed by fluid flow from outlet 104 to waste chamber 105. The high-throughput chip may perform differentiation and screening of neural stem cells (NSCs) for 12X8 cell chambers. In some implementations, chip 100X may generate drug gradients using convective-diffusion channels. and are entered in separate eight reactor arrays. The outlet of each concentration array is connected to the waste chamber by separately so that one concentration array will not contaminate the other. In some implementations, each concentration array may be connected to the waste chamber by a microchannel. Eight repeat experiments can be carried out in each toxin or drug concentration.

In some implementations, the high-throughput chip may be a multi-layer fluidic chip comprising a reaction layer having a plurality of microfluidic reactors, wherein fluidic perfusion is preformed in at least one of the plurality of microfluidic reactors, a microelectrode array layer under the reaction layer, the microelectrode array layer configured to stimulate at least one of a plurality of cells using at least one stimulating electrode, and sense data from the at least one of a plurality of cells using at least one sensing electrode. In some implementations, the fluidic chip may include a first layer having spiral channels for lateral flow of cells or fluids, a filter layer under said first layer for separating the two or more types of cells, a first co-culture layer for stimulating a first type of cells, and a second co-culture layer for stimulating a second type of cells. In one implementation, one of the two or more types of cells may respond to a chemical stimulation, an electrical stimulation, an optical or illumination stimulation, a fluidic shear stimulation, etc.

FIG. 1C shows a high-throughput screening chip with separate perfusion channels. These perfusion channels 110 can be fabricated in another layer of the chip with vertical connection channels and a filter layer across the vertical channel for securing the cells from perfusion channel flow, cells may enter through separate inlets 106. Perfusion reagents, such as drug and cell media, may be delivered into cell reactors, such as reactor 113, using pneumatic control through binary split channels, such as channel 107. The perfusion reagents may flow into inlet 106 and through the binary split channels, such as channel 107, and subsequent channels until being delivered to a reactor, such as reactor 113, through a reactor delivery channel, such as channel 108. In some implementations, the reactor delivery channel may deliver the perfusion reagents to the side of reactor 113 using fingers 110. Cells may be inserted into reactor 113 through inlet 111. In some implementations, the perfusion reagent may flow out of reactor 113 and out of the chip through outlet 109 to waste chamber 105.

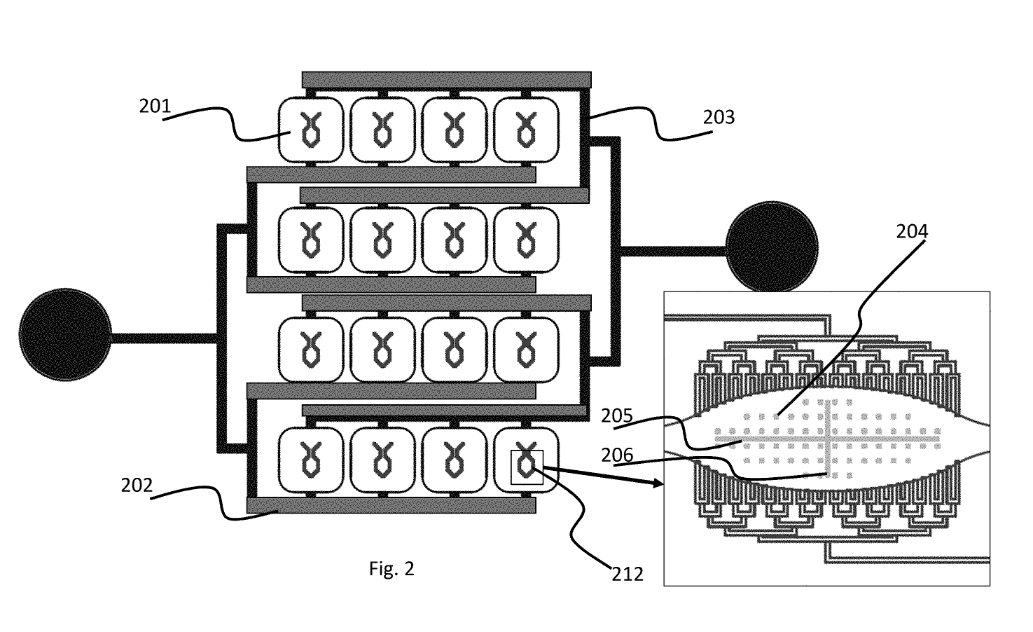

Multiple reactors 201 may be accessed by larger width channels horizontally 202 or vertically 203 to avoid fluidic resistance that may hinder uniformity in the flow of fluids to each reactor, such as reactor 113, as shown in FIG. 2. The bioreagents and the cells do not contact the manifold avoiding any contamination. In this chip, a plurality of cells may be loaded in each reactor, such as 100 cells, 200 cells, 500 cells, 1000 cells, 5000 cells, etc. Perfusion or fluidic pulse of cell media with nutrients may be performed for a period, such as several hours, several days, or several weeks. In some implementations, electrodes 204 may be used to measure impedance and/or field potential. Reactor 212 may include one or more ground electrodes. In some implementations, ground electrodes may be positioned within the chamber, such as horizontal electrode 205 and vertical electrode 206, where horizontal refers to electrodes oriented in the direction of flow of cells entering and exiting the reactor chamber, and vertical refers to a direction substantially perpendicular to the horizontal direction, and substantially parallel to a line connecting inlet fingers 110 and outlet fingers 114 of the reactor chamber. In other implementations, reactor 20X may include two or more vertical electrodes, two or more horizontal electrodes, and/or one or more electrodes oriented in a direction other than vertical or horizontal.

FIG. 3 shows a diagram of an exemplary serial high-throughput reactor. As shown in FIG. 3, the serial high-throughput reactor includes primary spiral channel 303 and a secondary spiral channel 304, where the inlets of the reactors, such as inlet 305, are connected to primary spiral channel 303 and outlets of the reactors, such as outlet 306, are connected to the secondary spiral channel. In some implementations, primary spiral channel 303 may have one or more inlets, such as inlet 301 and inlet 302. In one implementation, a concentration gradient on the serial reactors may be carried out in serial high-throughput reactor 300 by ramping the flow rates of two or more fluids in an increasing flow rate for one fluids and decreasing flow rate for the second fluids or vice versa. A combination of linear or non-linear orders may be used to create the concentration gradient in serial high-throughput reactor 300. The excess fluids may be collected at the outlet 307. In some implementations, each reactor may include one or more microelectrodes 308. Microelectrodes 308 may be used to measure impedance, field potential, and/or one or more intermittent optical signals.

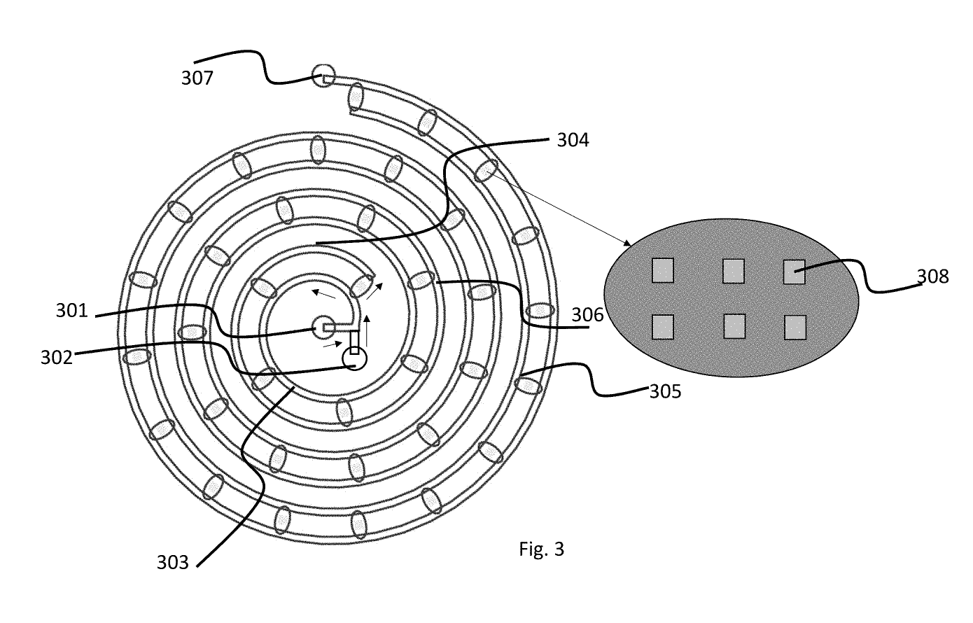

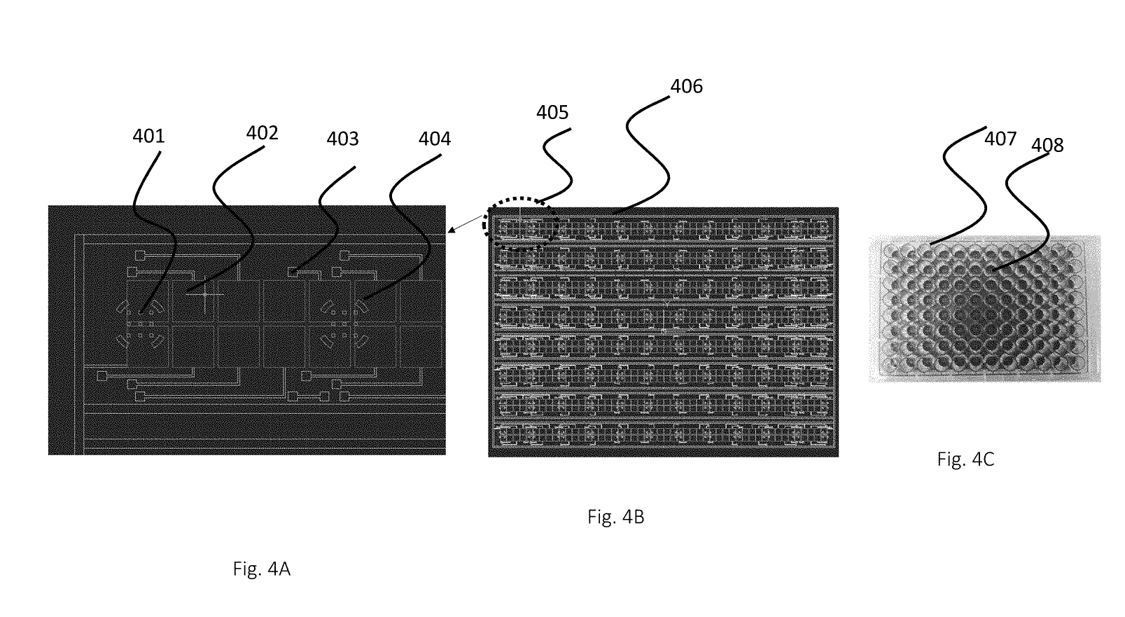

In each reactor, several recording electrodes and a center stimulating electrodes are formed in an n×m array 401 as in FIG. 4A. An electrode forming a ring around the array electrodes will be used as ground for stimulus and recording electrodes. The pads 402 for contacting external amplifier may be exposed on the bottom of the circuit board and vias 403 connect the bottom layer circuit to the top layer circuit. Initially, the chip may be made using an acrylic/printed circuit board layers with channel widths of 200 um and depth of 150 um and gold electrode (ENIG) of width 100 um with electroplated PEDOT.

FIG. 4B is the electrical sensor circuit 406 for the 96-well plate shown in FIG. 4C with wells 407 and electrodes 408. A portion 405 of the electrode array is shown in FIG. 4A. In some implementations, the fluidic dimensions may be optimized for low cost fabrication and flow behavior with an open-well plate, such as well plate 407. Well-plate 407 may be a 96-well plate. Each well of well plate 407 may be positioned over a set of electrodes, such as electrodes 401. Electrode set 401 may correspond to electrodes in n×m array 401. The ground electrodes are position in the corners 404. The device may consist of an upstream concentration generation module connected to an array of downstream cell differentiation reactors. The electrodes may be fabricated on transparent glass or plastic as single layer, or opaque printed circuit board as two layer with sensing electrodes on the top layer and contact pads on the bottom layer. Impedance, field potential measurements and/or electrical stimulation may be carried out.

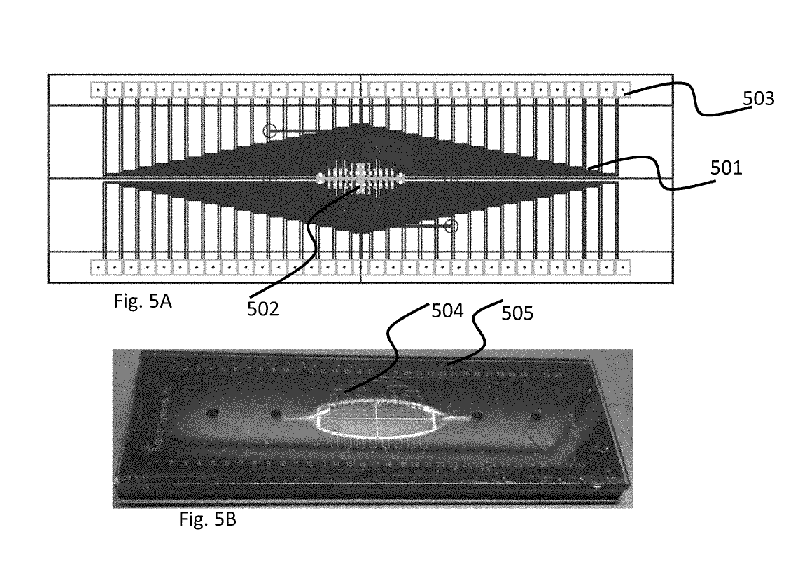

FIG. 5A shows a diagram of an exemplary reactor chip. The reactor chip includes a main chamber fitted with electrodes 501. Electrode sensors 502 may be positioned within a chamber of reactor chip 500A, and pads 503 for spring loaded connectors are positioned along the edges of the reactor chip. In some implementations, cells may enter into the chamber through cell inlets that are closed by a pin valve while cell media mixed with a chemical such as toxin, cytokines, stimulators or a drug such as that used for neurological diseases is perfused through multiple fingers though a set of valves at the inlet and outlet as shown in FIG. 5A. Cell culture is performed on a chip using one set of ports for cell entry and another set of ports for perfusion fluidic entry.

FIG. 5B shows a PCB based chip with transparent channels 504 and electrode pads at the bottom 505. Channels are made using acrylic layers while activation of fluidic shear stress is caused by on-chip PZT benders.

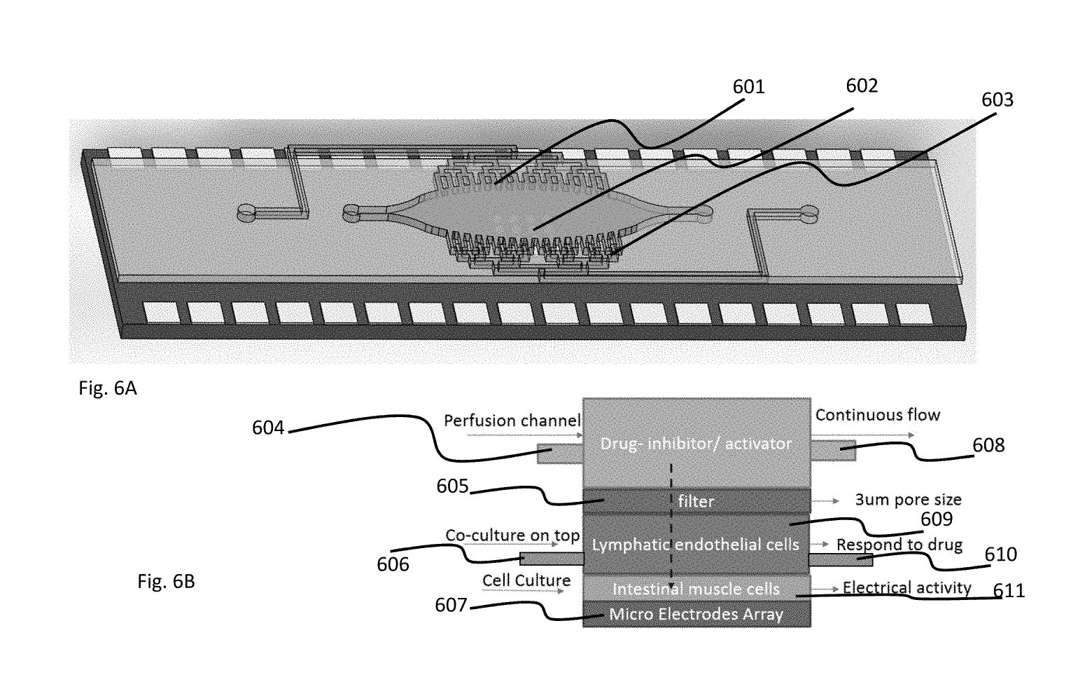

In some implementations, a coculture of two or more cells, such as neural cells, cardiac cells, muscle cells or any other cells within a fluidic chip 601 with electrodes 602 and perfusion channels 603 as shown in FIG. 6A.

These cell culture chips may have inlets 604, 606 and outlets 608, 608 on two or more layers of fluidics separated by one or more filters 605 of sizes depending on the cells used diffusion or convection of fluids within the system. In some implementations, filter 605 may have pore substantially uniform pore sizes of about 1 micro meter, 2 micro meters, 5 micro meters, etc. Drug study across lymphatic endothelial cell tight junction can be performed as shown in FIG. 6B using intestinal muscle cells 611 reading electrical activity from bottom electrodes 607.

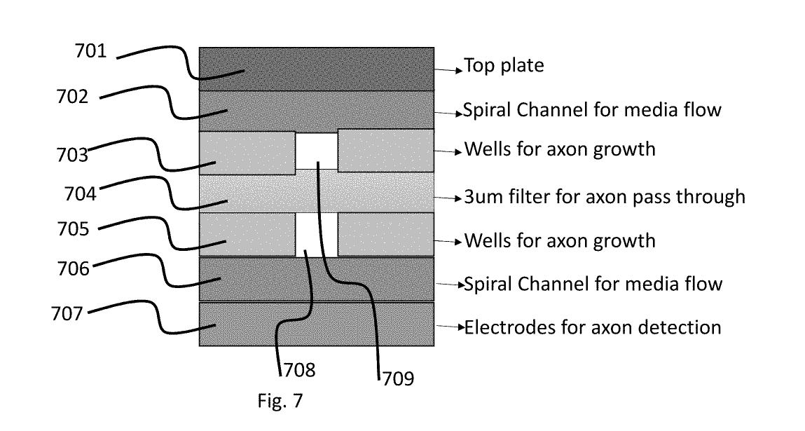

Further monitoring cells within a co-culture system using impedance or field potential measurements on electrodes 707 are performed for axon growth 709 through filter 704 between channels 709, 708 are reactors as in FIG. 7. In some implementations, the channels may be spiral channels or linear channels. Screening of drugs or toxins or differentiation of pluripotent stem cells are achieved using two or more cell types within two or more compartments of fluidics separated by filters.

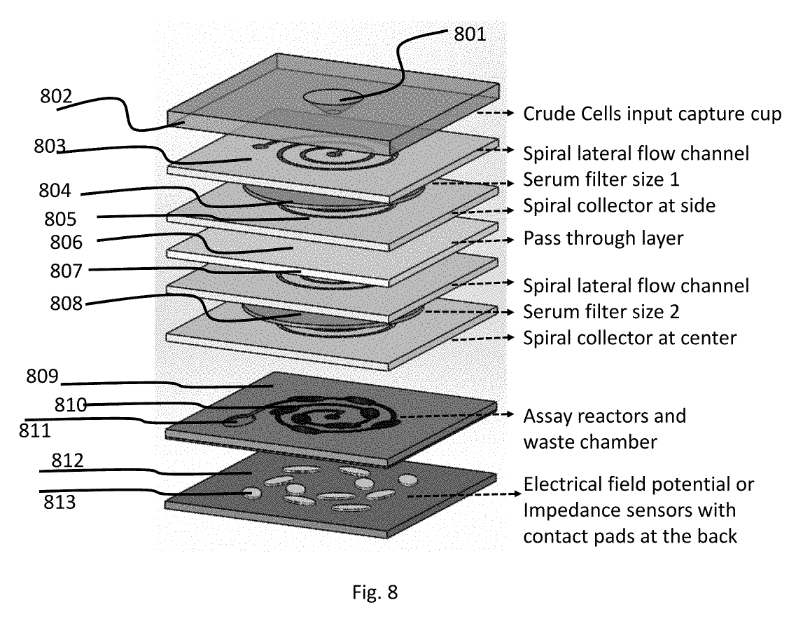

Performing coculture of cells in channels with ellipsoidal shaper or spiral shape channels are developed (FIG. 8) where separation of a cell population from mixture of cells at the inlet 801 are carried out using successive size-based separation in spiral fluidic channels 803 and 805 separated by filters 804 and 808 of increasing sizes. Placing filters 804 and 808 between spiral channels 803 and 805 may help in the effective filtration process under laminar flow.

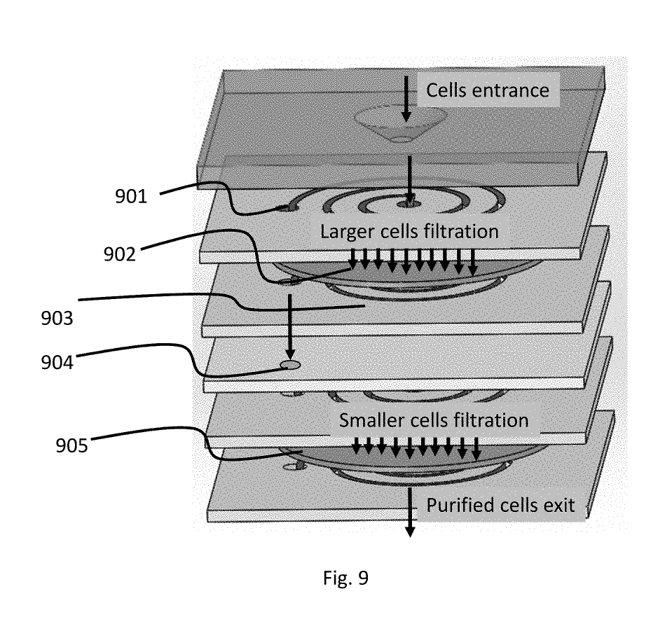

Larger cells may be filtered using filter 902 and smaller cells may be filtered using filter 905, as shown in FIG. 9. Additional layer 904 may be positioned between the two-filter system to pass fluids between filters.

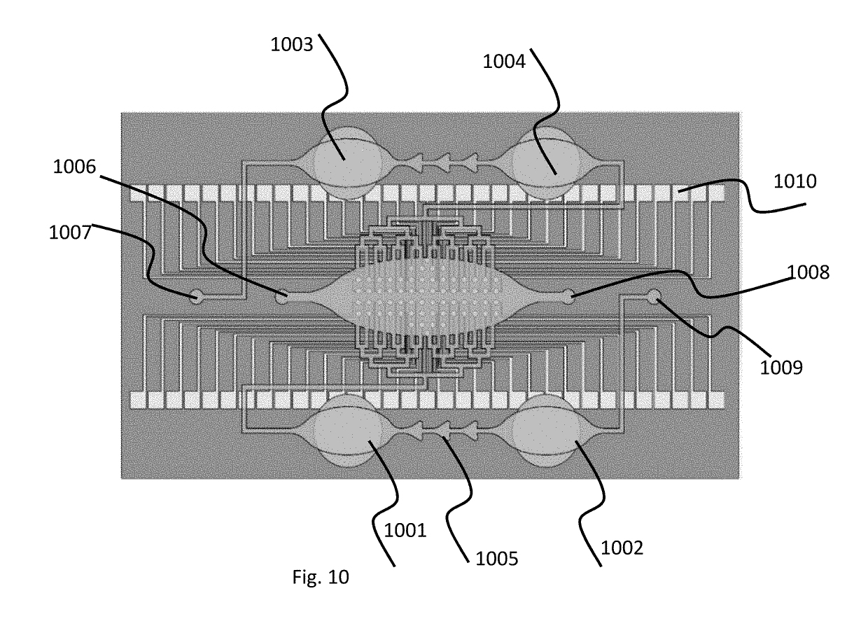

Fluidic shear stress may be imposed on the cells using a pair of piezo (1003, 1004) and (1001, 1002) or peristatic recycling pumps as in FIG. 10. A diffuser nozzle valve 1005 is connected in between the piezo pumps. These mechano stimulations of cells using fluidic shearing is performed through pumps integrated on chip or through external pumps.

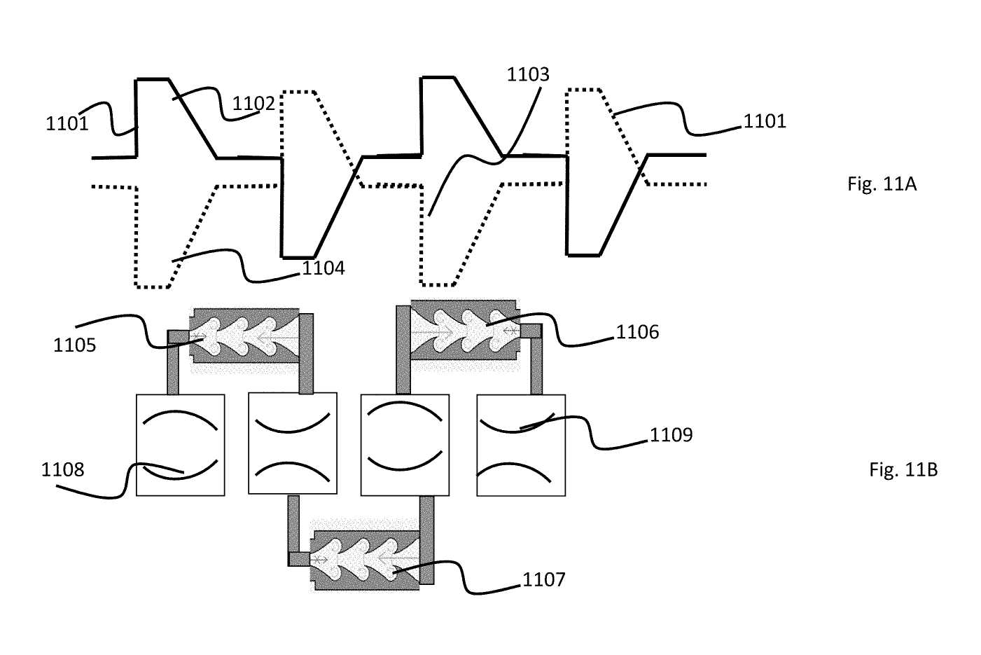

Pumping fluids using pairs of piezo electric actuators (PZT) blenders is carried out using AC voltages in trapezoidal wave form 1101, 1102 (FIG. 11A) in cascaded or peristaltic modes of operation. The solid line 1101 and dotted line 1104 show the ac pulse for two sandwich PZT disks with fluids for pumping. With tesla, diffuser/nozzle valve 1105 or combinations 1106, 1107 separating different stages (FIG. 11B) to direct the pumping. The PZT disk on the application of the electric signal bends concave 1108 or convex 1109. The perfused media at a waste chamber will be collected for further excreted protein analysis. In this chip, perfusion or fluidic pulse of cell media with drug is carried out for several days to weeks.

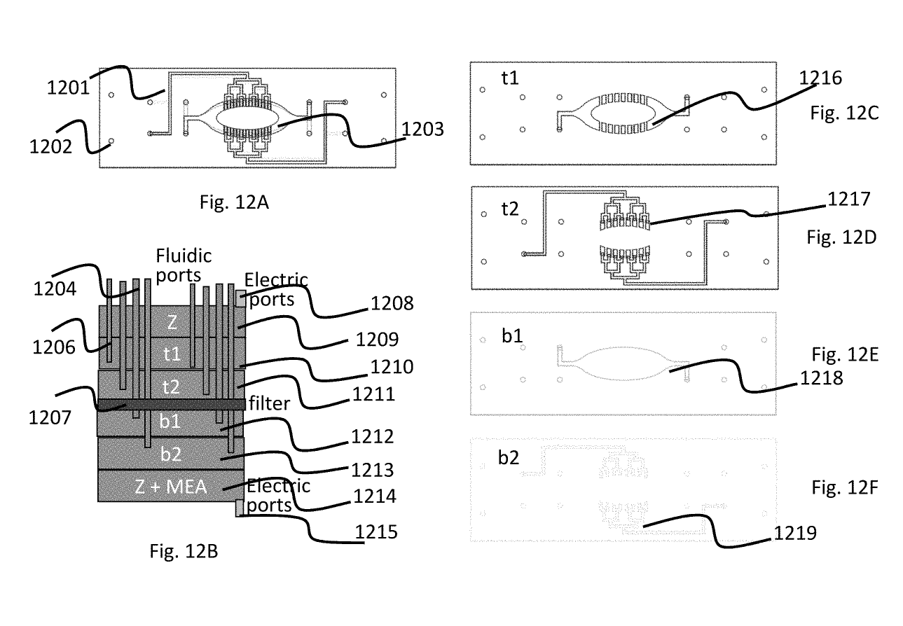

Fabrication of co-culture cell chip to perform endothelial tight junction based cell assay using multiple layers of channels 1201 are developed as in FIG. 12A-FIG. 12F. The corner holes 1202 are for alignment and attaching to fluidic manifold and cells can be in a ring type channel 1203, 1216 on the top layer so that the bottom layer can be visualized. These reactors are equipped with multiple electrodes 1208 and 1215 for monitoring cell growth or cell behavior using electrical impedance measurements and/or field potential measurements 1214 within each compartment or between the compartments. Further fluidics ports 1204 to flow fluids on the top layer 1210, 1211 as well as in to the bottom layer 1212, 1213. A ring filter 1207 is attached between the top and bottom fluidics to keep the cells within compartments and grow the cells on it. The top 1217 or bottom channel 1219 can have fluidic fingers.

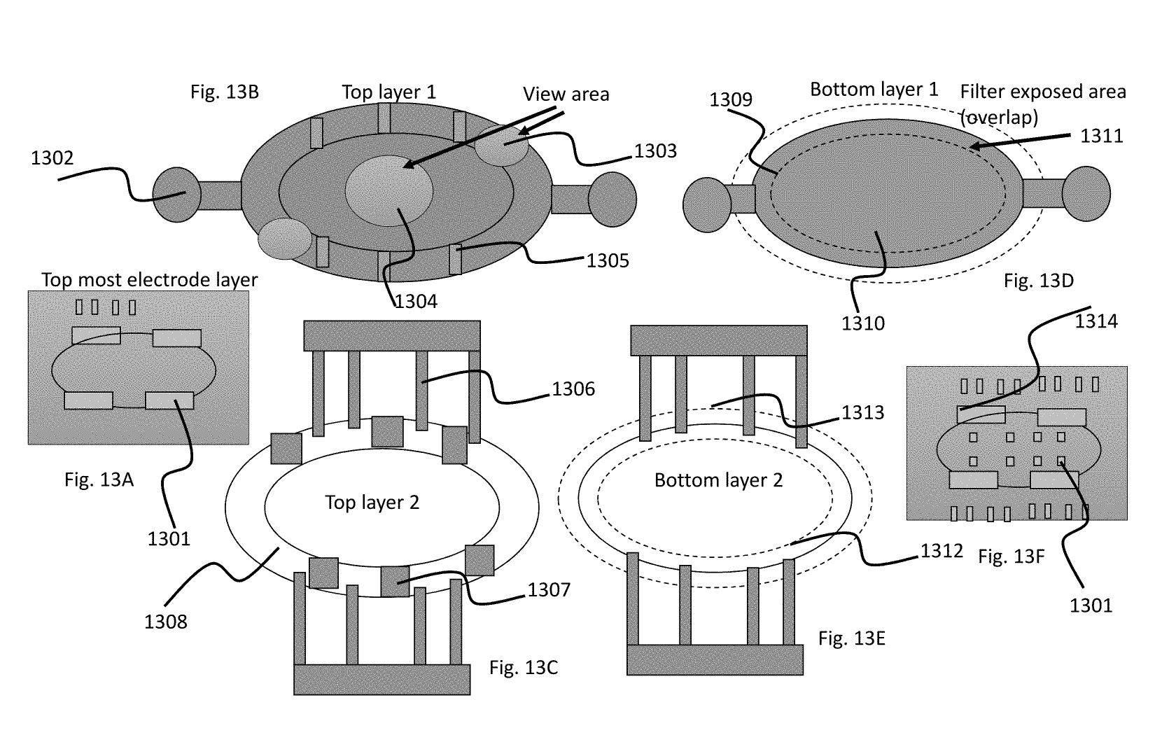

The top 1301 and bottom 1314 layer can have multiple electrodes for impedance electrodes in quadrants for measurement across top and bottom layer as in FIG. 13A and FIG. 13F. The field potential electrodes 1301 are also included in the bottom layer. The top layer has opening 1304 or transparency 1303 for viewing the cells as in FIG. 13B. The finger channels 1306 will direct fluids normally to another set of channels 1307 as in FIG. 13C and FIG. 13E. There is an overlap 1311 in the top 1309 and bottom chambers 1310 to expose the filter and allow diffusion of fluids as in FIG. 13D.

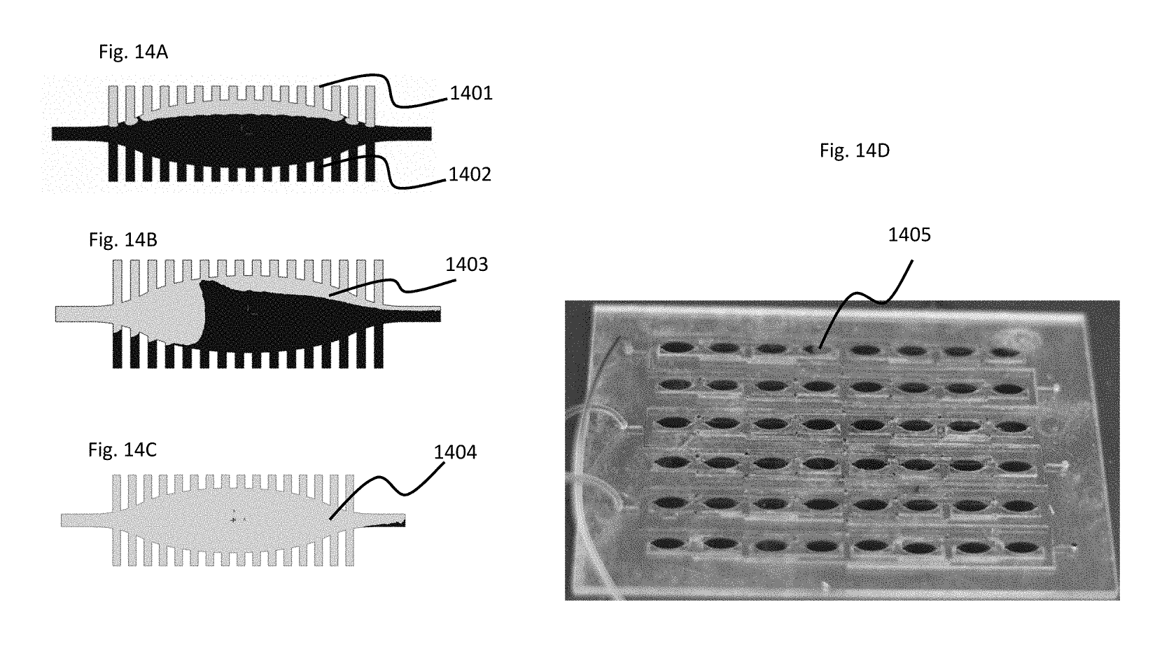

Pumping and filling fluids or priming in a chamber using three step operation by priming 1403, 1402 the side finger channels before cells input, filling the chamber with cells through main inlet and to continue perfusion through the side fingers 1401 are performed as in FIG. 14A to FIG. 14C. FIG. 14D shows a high-throughput chip with 48 reactors 1405.

Stimulations and Recording Electrodes:

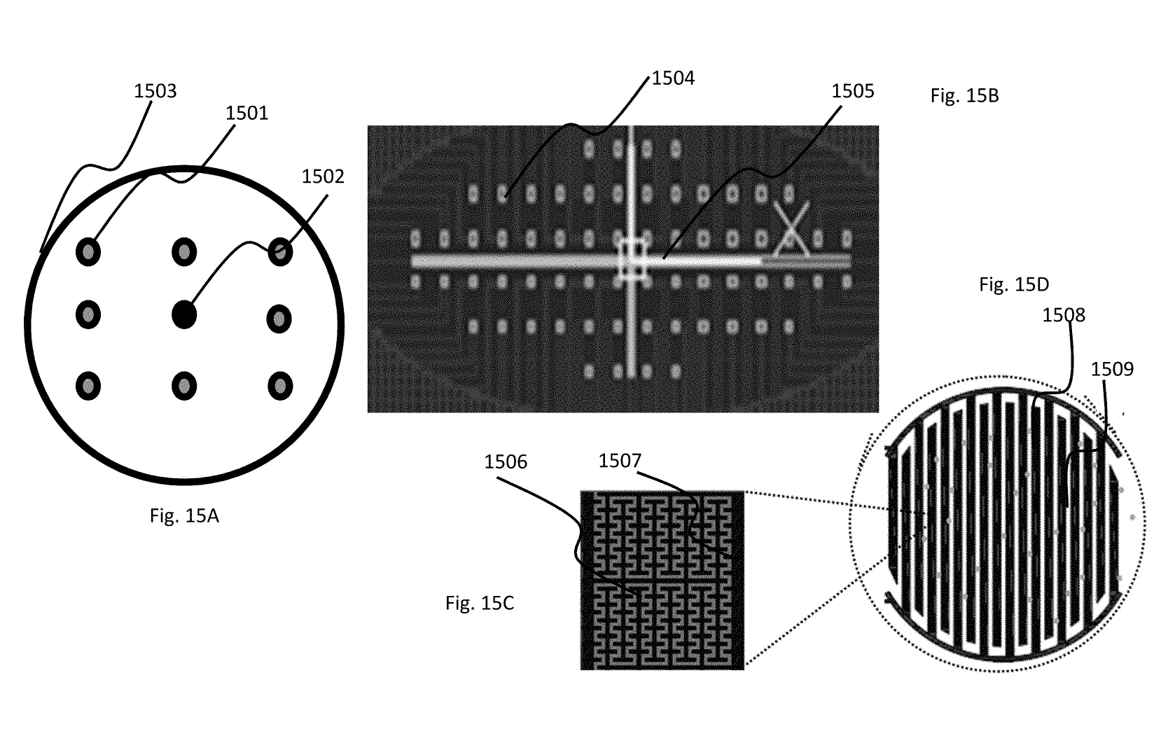

In each reactor, several recording electrodes 1501 and a center stimulating electrode 1502 are formed in an n×m array as in FIG. 15A. For large arrays configuring electrodes for stimulation is carried out by choosing electrode 1504 with cells or middle electrode 1505 and monitoring cells using an array of electrodes configured in the flow path of an elliptical reactor as in FIG. 15B. Monitoring cells using fractal electrodes 1506 (FIG. 15C) or interdigitated electrodes 1508 (FIG. 15D).

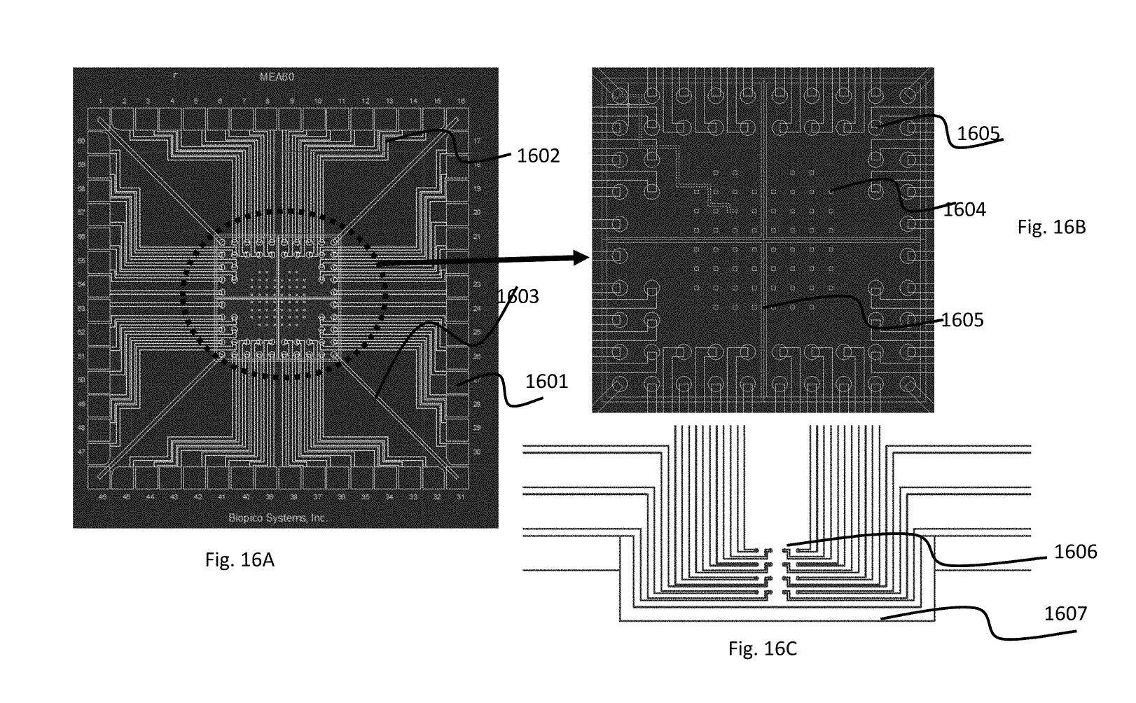

In circular reactors, electrode array is configured as in FIG. 16A. Electrodes connectors 1602 and pads 1601 are arranged in orthogonal while corner electrodes 1603 is arranged diagonally.

FIG. 16B shows the location of ground electrode as cross 1605 in the middle of the chip separating quadrants of the chip. The electrodes sensors 1604 are connected to pads using vias 1605 connections. Outside the sensor electrode tips 11604, 606 are covered with solder mask.

FIG. 16C shows long ground electrode 1607 on a side of the chamber.

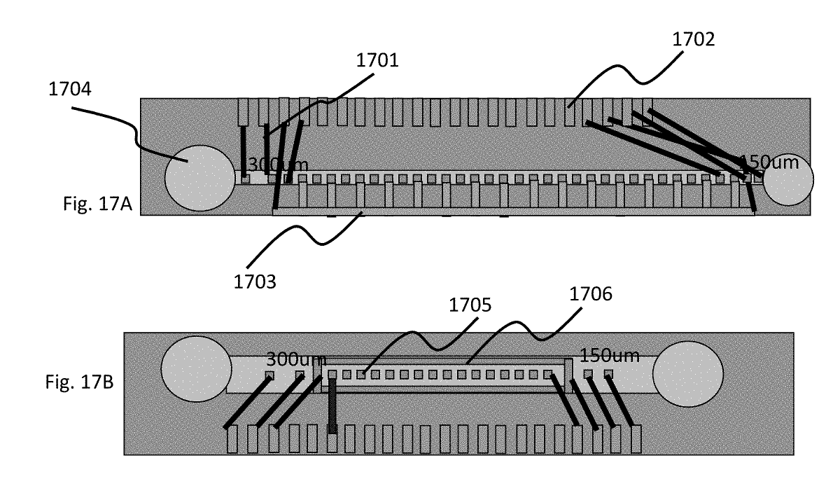

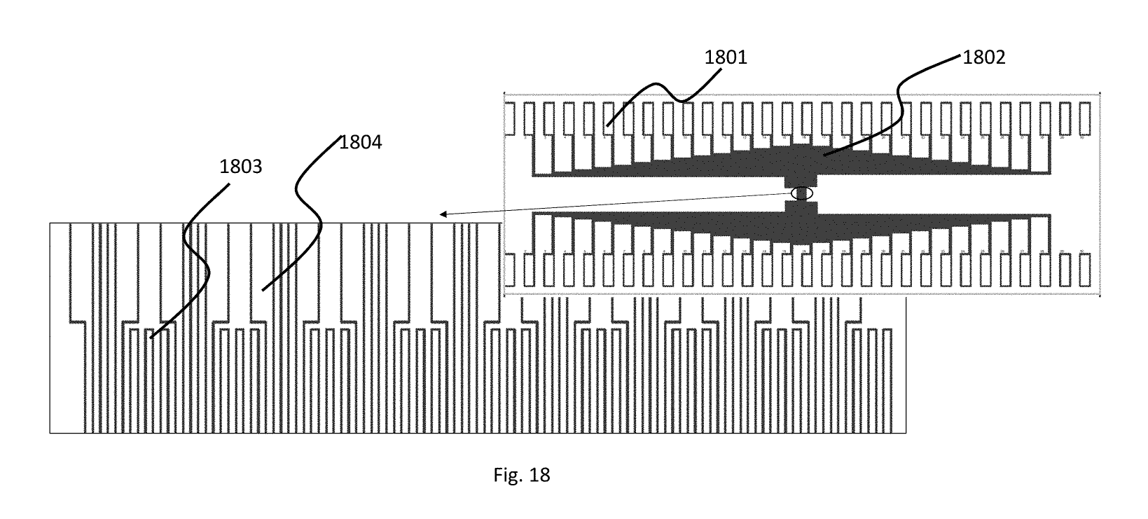

Configuring channels in a one-dimensional (1-d) format is carried out either as single ended electrodes 1701 with reference or ground electrodes 1703 on a side (FIG. 17A) or as a single ended electrode 1705 with vias on each electrode (FIG. 17B) so that ground or reference electrodes can be around 1706 them or as differential electrodes (FIG. 18).

The differential electrodes 1802 will have triple electrodes with two fat electrodes 1804 sandwiching a thin electrode in the middle 1803. Generating electrical stimulus signals with biphasic pulse train or monophasic with positive or negative pulses with interphase delays with a phase difference between the pulses shaped as square wave form.

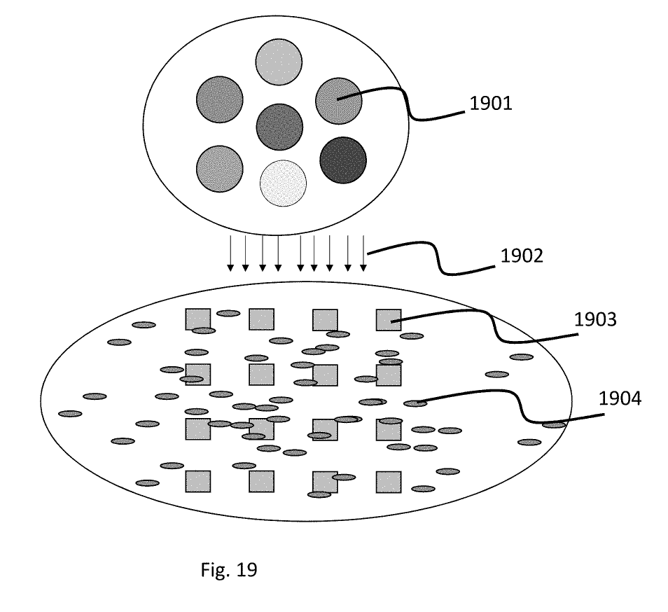

Generating optogenetic stimulation 1902 on cells 1904 using light of a particular wavelength 1901 and magnitude with a fixed period or wave form is carried out coupled with electrical stimulation as in FIG. 19. Field potential signals are picked up by electrodes 1903.

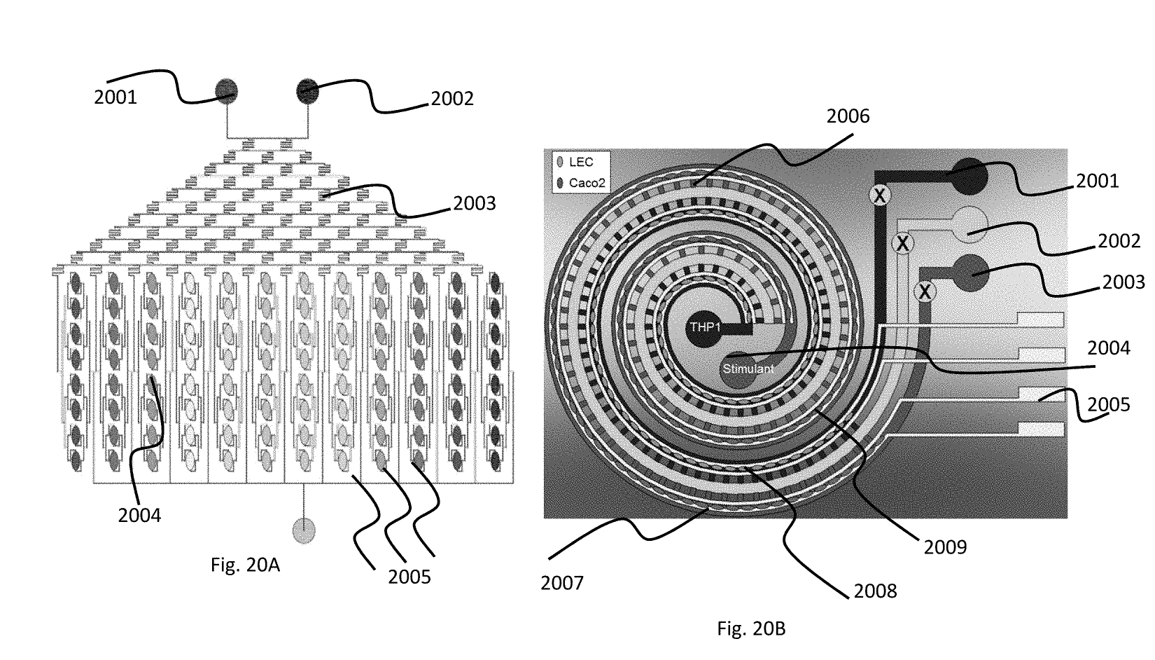

Stimulating cells using chemicals or combinations of chemicals 2001, 2002 for a fixed period of fluidic pulses or constant perfusion flow using a specific concentration 2004 or gradient of concentrations 2005 or composition of chemicals across chambers is shown in FIG. 20A. Chemical stimulations across multiple concentric spiral 2007, 2008 micro/nano spaced interconnected channels where multiple inlets 2004 and outlets 2001, 2002, 2003 of the spiral channels serve as inlets and outlets to stimulant or cells 2009 forming tight junctions to transport fluids across multiple channels for drug screening application assessed by trans-epithelial electrical resistance measurements 2005 are shown in FIG. 20B.

Nanostructured Electrodes:

The bottom glass plate with indium tin oxide (ITO) electrodes coated with nanostructured gold or platinum or electroactive polymers such as Poly(3,4-ethylenedioxythiophene)-poly(styrenesulfonate) (PEDOT:PSS) forms the basis of electrodes for electrical stimulation and monitoring. In these electrodes cell lays completely tight improving the electrode cell interface impedance to acquire signals at high sensitivity.

### Design and Development of System

Fluidic Perfusion:

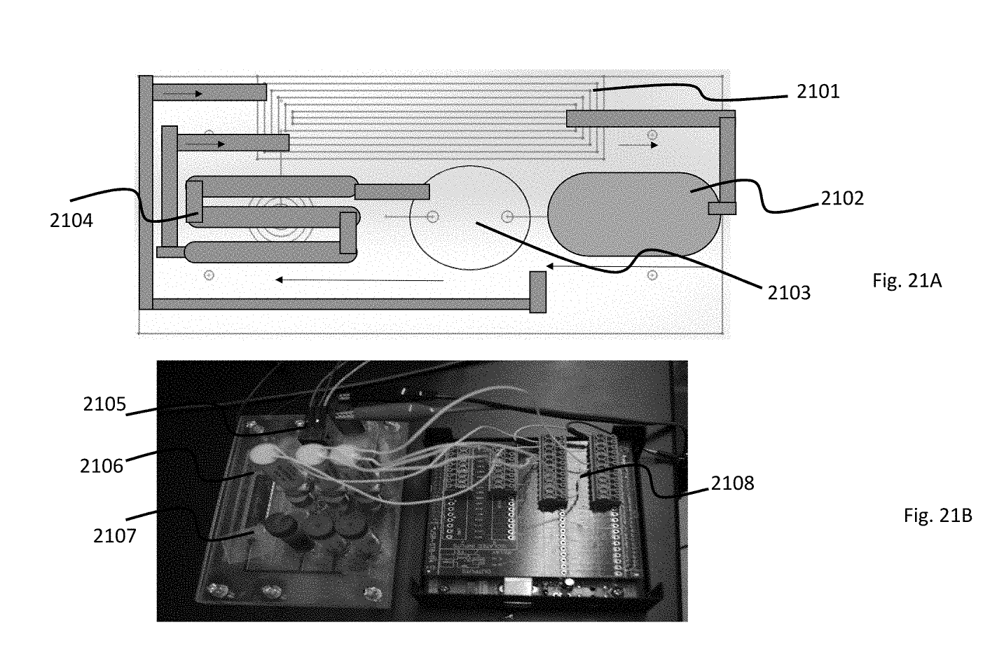

The system performs high-throughput analysis of neuronal drug in microreactors using microelectrode array electrophysiological signatures. In some implementations, the system may include a 96-well format with gradients for drug/combinatorial doss produced by a microfluidic network and repeated reactors. The system is configured to perform fluidic perfusion, cell stimulation and data acquisition from multiple microelectrodes in the reactor. Methods for building a manifold using serpentine 2104 or ellipse channel or cylindrical reservoirs (FIG. 21A) to a reactor 2101 and pull the fluids to waste using a vacuum pump 2103. A pressure sensor 2105 monitors pressure of the system and a set of valves 2106 release fluidic pressure from a set of reservoirs 2107 using is shown in FIG. 21B. A relay 2108 is used to activate the valves and pumps. The cells are captured at the bottom channel and any unused cells are collected at the waste outlet. TruStability® High Accuracy Silicon Ceramic Series piezoresistive silicon pressure sensor (Honeywell) is used to measure the hydraulic pressure across the channel.

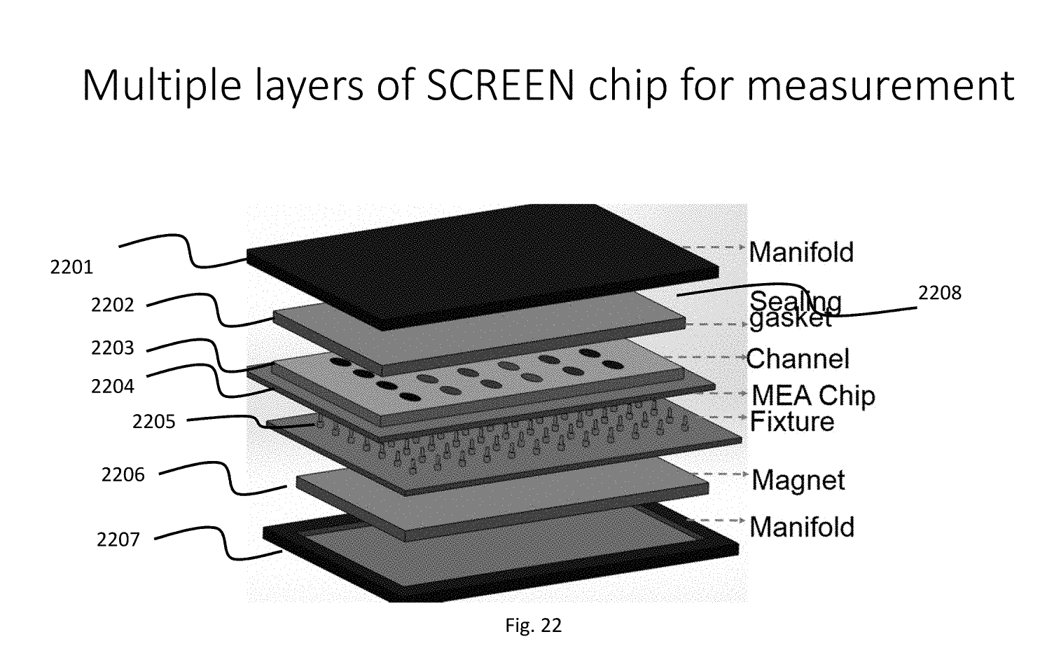

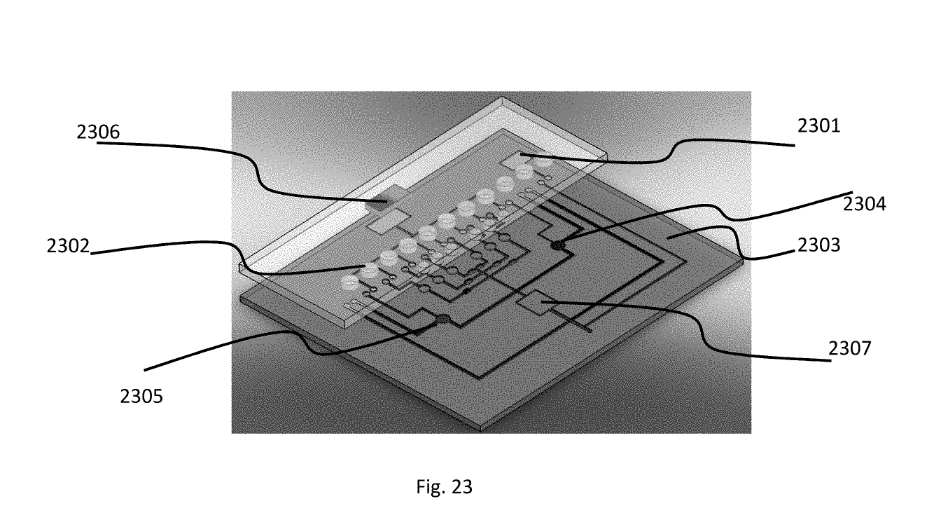

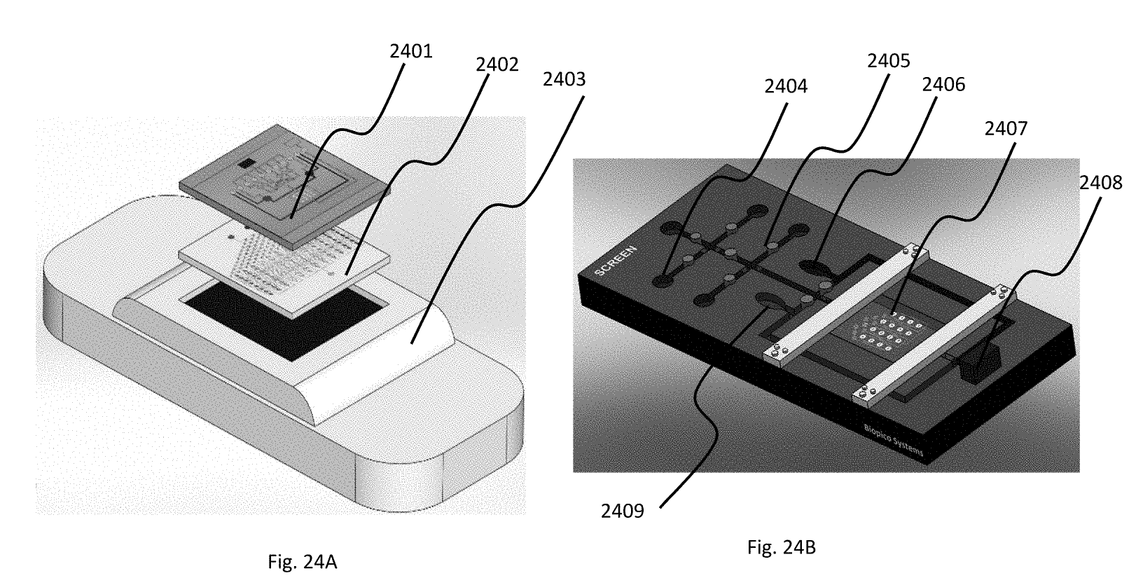

The fluidic chip 2203 docks in a fluidic manifold 2207 and an electrical fixture 2205 using a pair of magnetic plates 2206, 2202 and a sealing gasket 2208 (between top manifold and top magnetic plate) as shown in FIG. 22. The reagents such as drug, cell media or buffer are stored in the disposable chip 2307 as in FIG. 23. The flow control is facilitated using an array of valves 2302 and pumps 2301 for delivering fluids into the channel with a flow rate between 1 μl/min and 100 nl/min. The cells are delivered manually at different reactors and perfusion of media/reagents will be carried out by electronic control. The fluidic manifold is set on top of the reactor wells to form an air tight seal using magnets. PCR can be carried out using hot 2305 and cold 2304 reservoirs on chip.

Initially, a microincubation system may maintain cells at a temperature of about 37° C. and about 5% CO₂, allow continuous medium perfusion, and prevent evaporation. As a second step, portable system 2403 may hold chip 2402 and manifold 2401, as in FIG. 24A with temperature/humidity tolerance instrumentation that can operate remotely in a lab incubator. The cells are delivered manually at the chip and perfusion of media/reagents will be carried out by electronic control. Operation of the pump 2408, sensors and valves 2405 will be controlled. At the end of the perfusion or differentiation experiments, RT PCR can be performed on-chip using hot oil 2409 and cold oil 2406 in reservoirs on manifold for gene expression profiling as in FIG. 24B.

Multimodal Stimulation:

The system will perform electro mechano chemical stimulation and long term monitoring using microelectrode array based electrophysiological signatures and optical imaging. The system is configured to perform fluidic shear stress, perfusion, cell stimulation and data acquisition from microelectrodes in the reactor. Optical stimulation is carried out using LED at different emission wavelengths. Further electroporation is carried out at higher currents on chip for transfection experiments or drug delivery into the cells. Acoustic/ultrasonic stimulation can also be carried out using integrated PZT or sealed PZT.

Electrochemical Sensing:

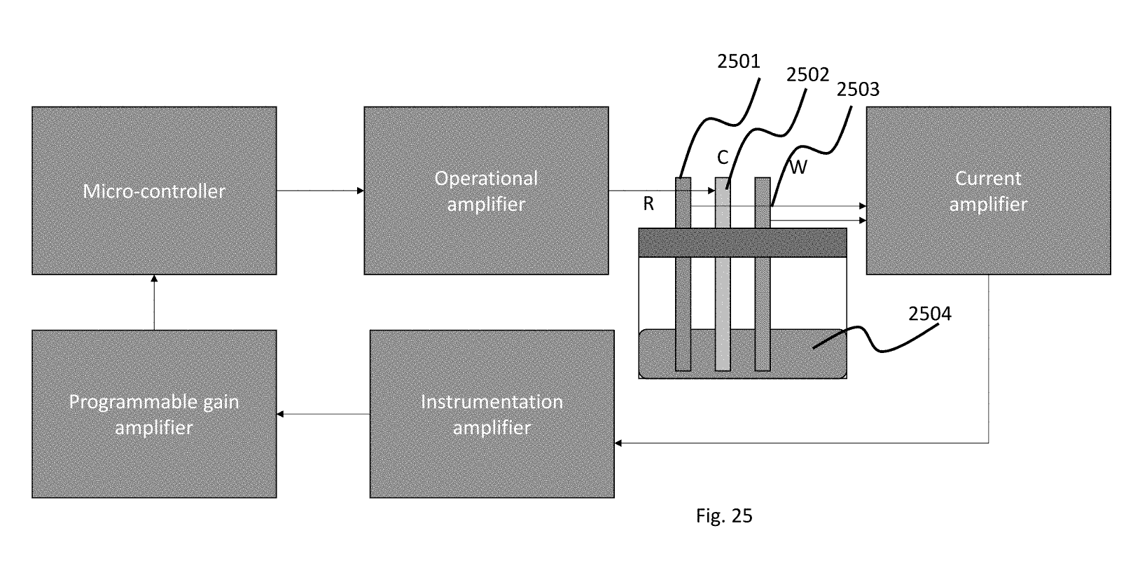

Electrochemical measurements using two or three electrodes 2501, 2502, 2503 in the reactors 2504 is presented in FIG. 25. Oxidation and reduction measurements can provide information on proteins secreted 2504 from the cells.

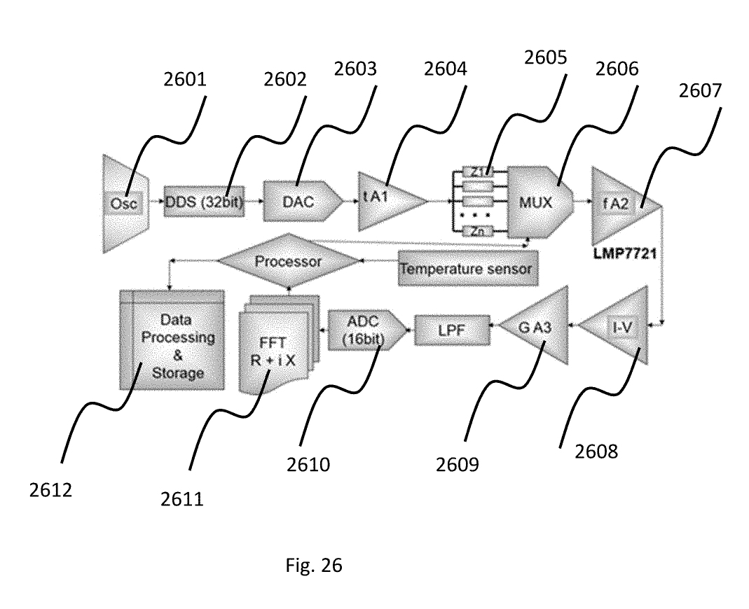

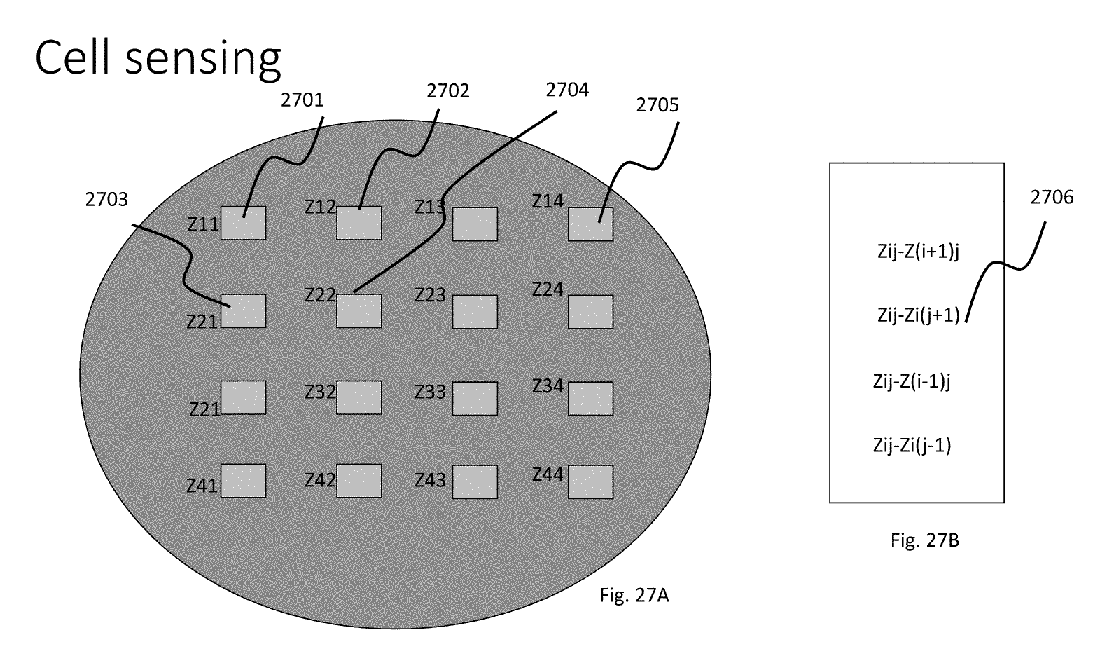

Further, non-faradaic high speed impedance measurements (FIG. 26) can provide information on the cells bound on the surface 2605. Multiple reactors with impedances Z1 . . . Zn can be measured using digital to analog conversion 2603, amplification 2604, multiplexing 2606 another amplification 2607 current conversion 2608 analog to digital conversion 2610 followed by Fast Fourier transform (FFT) 2611. For example, cardiomyocytes beating is tracked from the high-speed impedance measurements from the cells under stimulation.

Further single cell impedance 2701 tracking from different electrodes 2702, 2703, 2704 can track the presence of cells on electrodes (FIG. 27A) by measuring 2706 differential electrodes from all sides as presented in FIG. 27B. Higher differential impedances from adjacent electrodes in all sides indicate that a cell is present at the vicinity of the electrode under investigation.

Electrophysiological Recording:

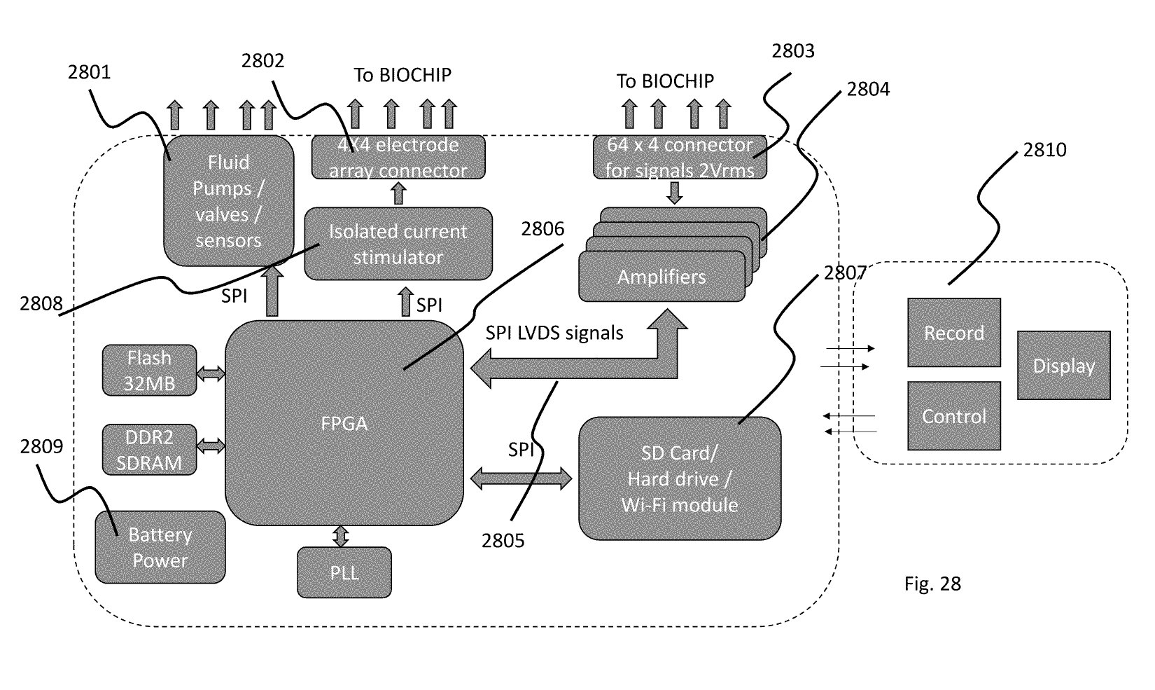

Analog signals from the cells are amplified using a low noise amplifier array 2804 and data are acquired at thirty-thousand (30,000) Samples/sec/channel using an FPGA 2806 through serial peripheral interface (SPI) to Low voltage differential signaling (LVDS) 2805 as in FIG. 28. On-demand field potential data acquisition from the cells 2803 is possible through wireless control and data transfer using the system. An isolated current stimulator array 2808 stimulates an array of electrodes 2802. Pumps and valves 2801 allows perfusion of fluids in the reactor and the entire setup operates from a battery 2809. The data will be wirelessly transmitted and record in a server using a front panel 2810 in a client machine.

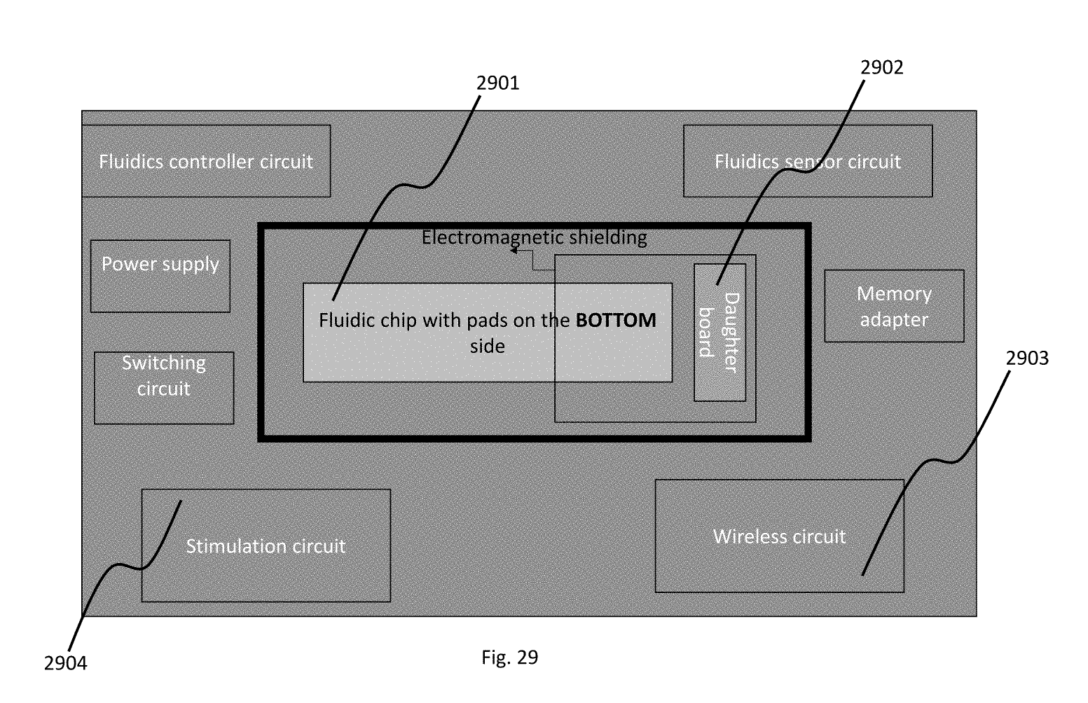

The recording electronics system has a double-sided PCB with electrical circuits with electromagnetic shielding of low voltage signals on one side and pads of bio fluidic chips 2901 on another side as shown in FIG. 29. The stimulation circuit 2904 and wireless data transmission circuit 2903 are designed in corners of the board. A daughter board 2902 to amplify the signals are connected closely to the biochip. The spike characteristics are recorded for every dose and monitored periodically remotely.

Multiparameter Sensing:

Multiple parameters such as temperature, humidity, pH, dissolved oxygen, CO2 level, and pressure values are monitored using sensors fitted in the bioreactor chip. In one implementation, the system may monitor the cell using electroporation based transfection of molecules for pharmacological screening.

Instrumentation and Control:

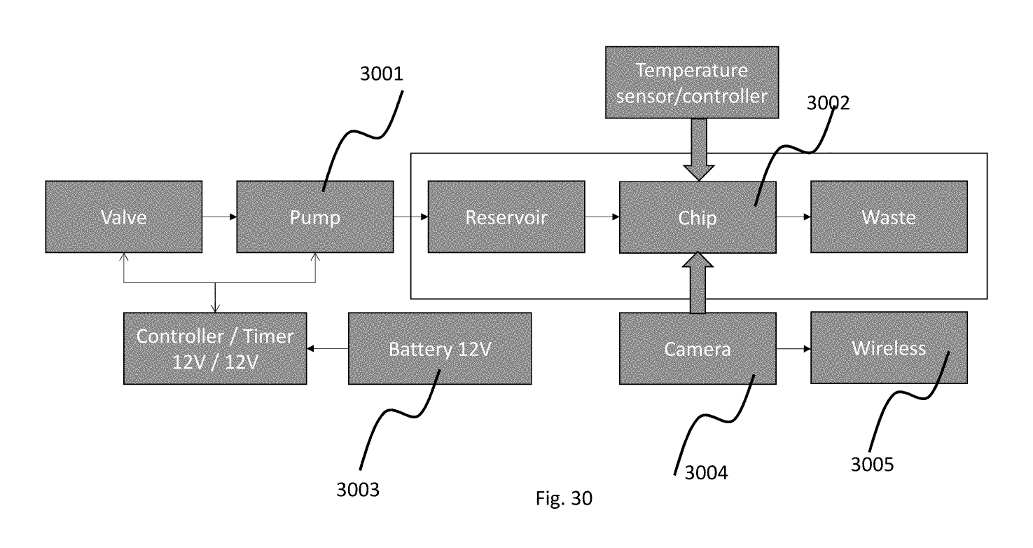

The system with pumps 3001/valves for a chip 3002 is controlled by wireless 3005 from a client terminal such as PC or smart phone for stimulation, recording or data transfer operated using a battery 3003 as shown in FIG. 30.

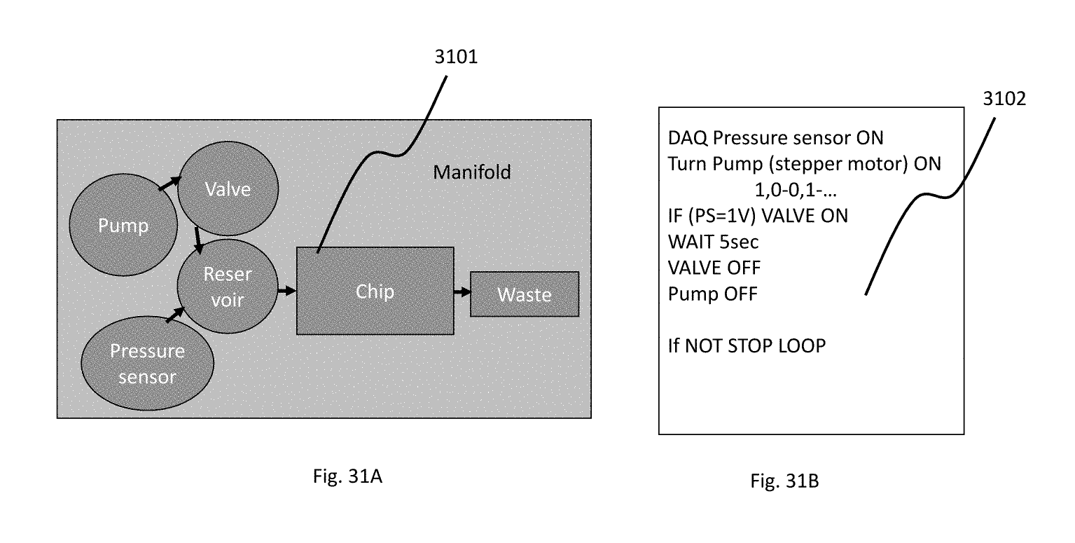

Fluid from a reservoir is flowed to the chip 3101 by pressure control and valve actuation as in FIG. 31A and the system is programed for automated operation as in FIG. 31B.

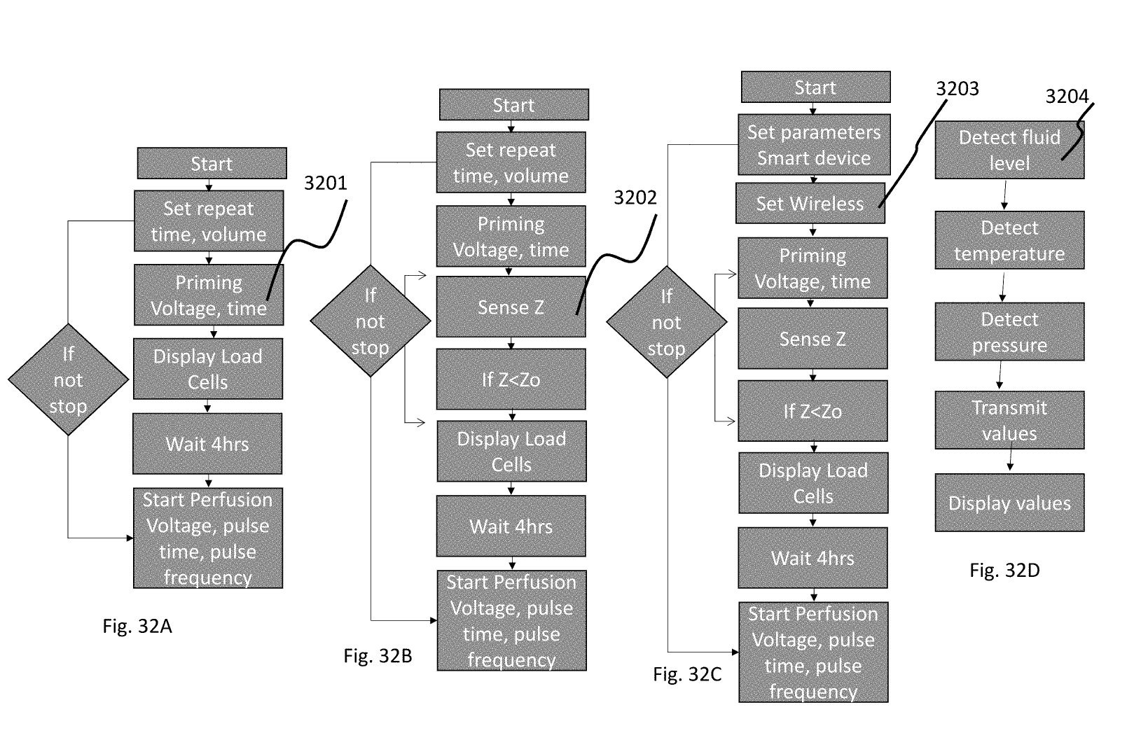

Further the fluidic control and cell monitoring can be automated 3102 as an open loop control (FIG. 32A) with controlled priming of channels 3201 or as closed loop with sensors such as impedance 3202 (FIG. 32B).

The entire system is made as a portable unit and is controlled remotely using wireless 3203 (FIG. 32C) with recording additional sensor parameters 3204 (FIG. 32D) for long term time controlled experiments.

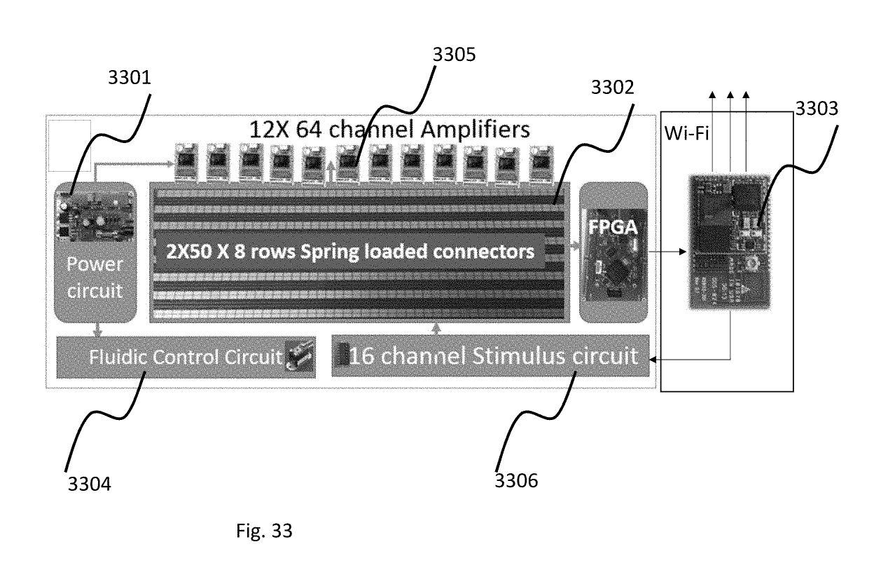

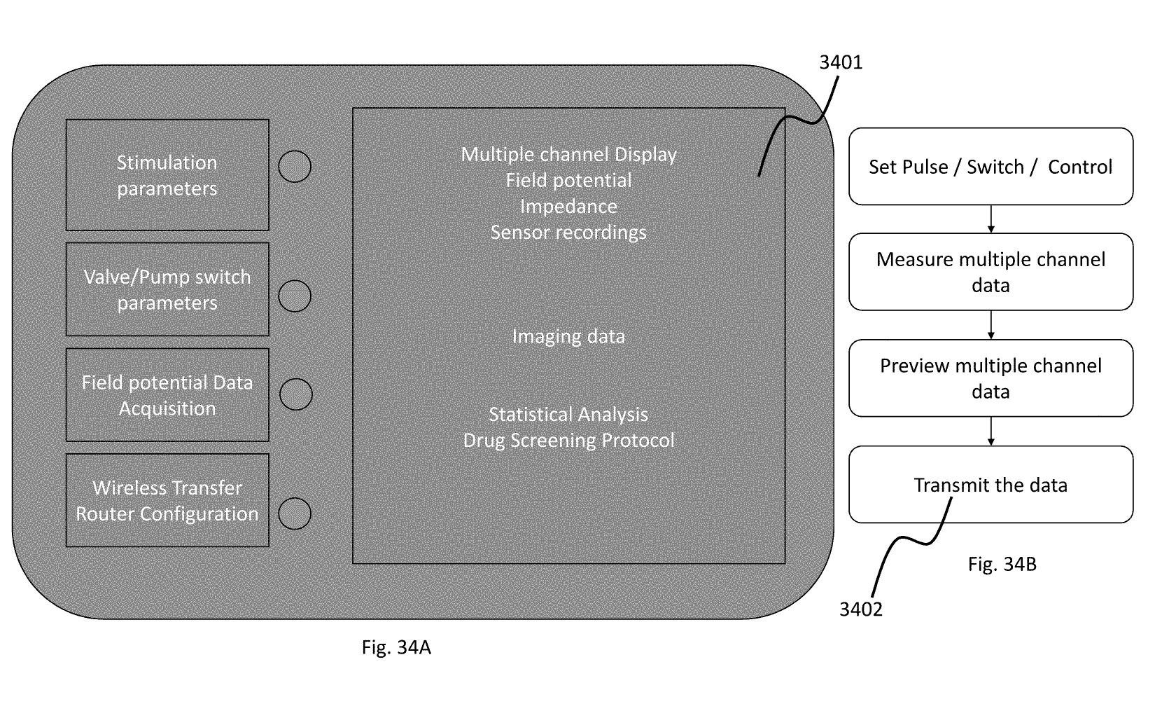

FIG. 33 shows high-throughput field potential or impedance measurement system from multiple electrodes using an array of cascaded amplifiers 3305 or circuits and stimulation system 3306 using an array of voltage/current generator circuits with wireless transfer 3303 of data and control.

Displaying data 3401 on a screening for user view and analysis and control of devices and storage or transmission of data 3402 for software development is shown in FIG. 34A and FIG. 34B.

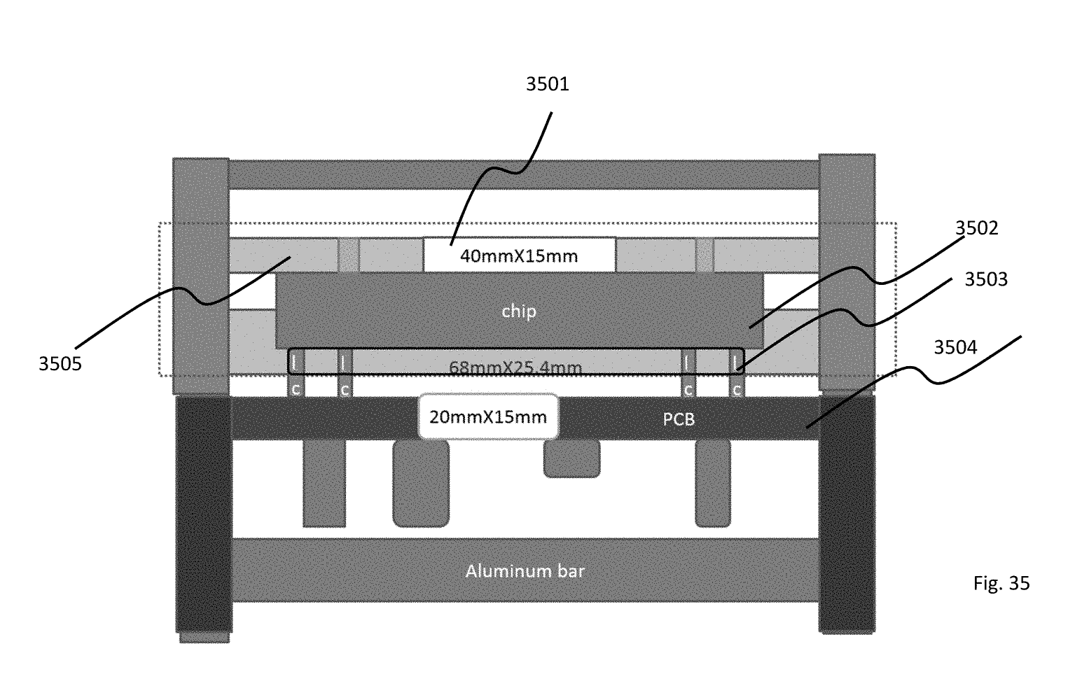

Mechanical design of the system for securing, communicating electrical signals suing spring loaded connectors 3503 and recording optical images from biochip 3502 using multiple layers of PCB fixture 3504, fluidic manifold 3505 with opening 3501 at the middle to optical transparency are shown in FIG. 35.

Statistical Analysis:

For the determination of statistical significance, 1-way ANOVA analysis followed by the Tukey's multiple comparisons test or Dunnett's post hoc test with appropriate control will be carried out. Data will be presented as mean±standard deviation. Drug effects will be evaluated using one-sample Student's t-test. A P value of less than 0.05 will be considered as statistically significant. The reliability of the measurement will be tested by multiple repetitions of the same experiment. Variance of the obtained data will be tested within the same culture and between different cultures.

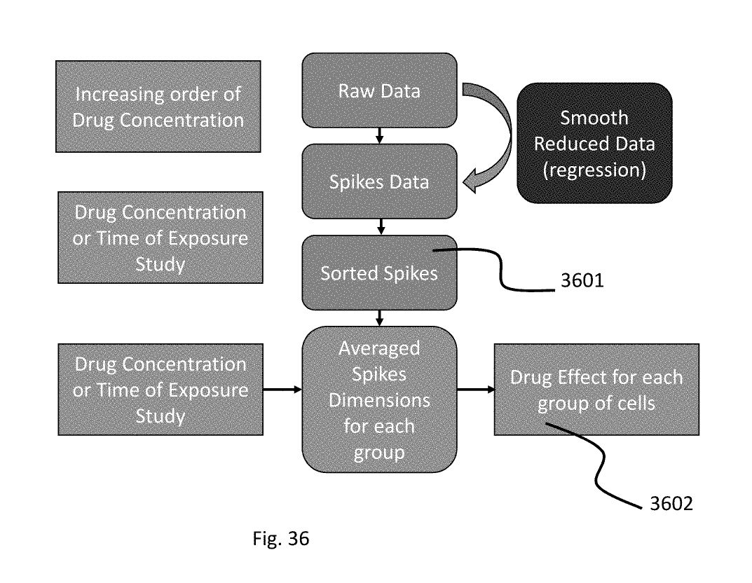

Post processing for images, electrophysiological data and pressure sensor data are performed using custom software. Drug study and analysis using field potential signal spikes sorted through spike sorting algorithms 3601 for uniformity of the cells under study to establish the effect of drug on the cells 3602 under study is shown in FIG. 36.

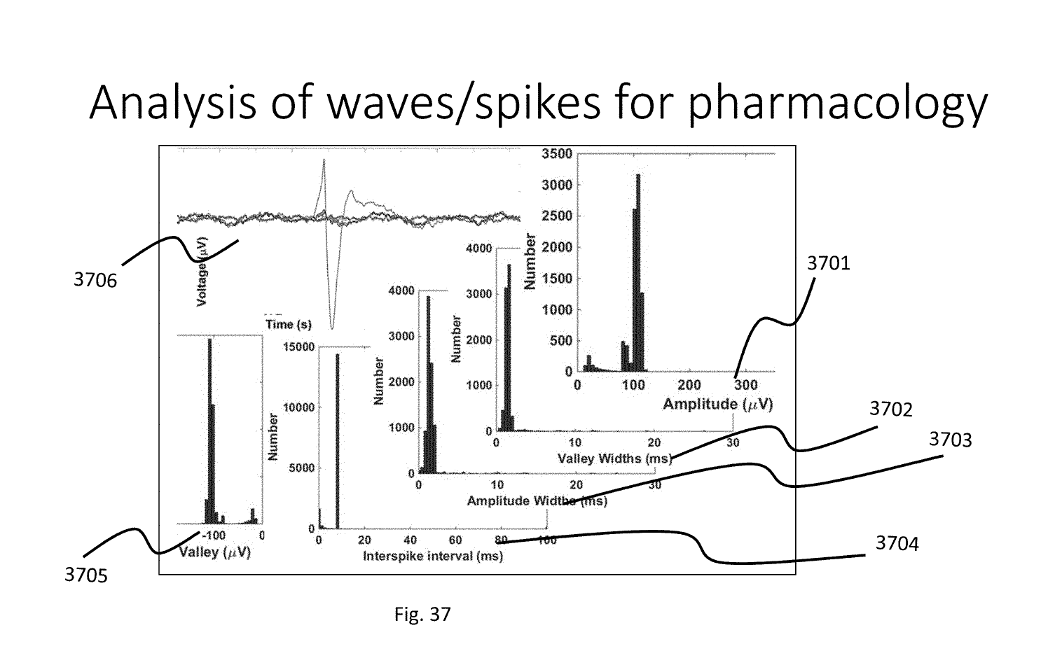

The spike characteristics as shown in FIG. 37 are recorded for every drug dose and monitored periodically. Spike detection and sorting is carried out using amplitude thresholding algorithm with a threshold set to 7 times the standard deviation of the filtered data. A spike 3706 was sorted into a cluster if the minimum distance was smaller than a threshold calculated from the noise of the filtered data. Several spike parameters including valley 3705 and peak amplitude 3701, widths 3702, interspike intervals (ISI) 3704, firing rates, and area under the curve are statistically calculated for a group of sorted spikes. Burst activity was characterized using an adaptive algorithm: (1) Computed mean interspike interval of all spike data (ISI₁). (2) Included spikes in subset with ISI<IS₁. (3) Computed mean ISI of this subset (ISI₂). Bursts were defined as consecutive spikes with ISI<ISI₂.

Excretion Sensing:

Analysis of secreted molecules from cells provides cues for proliferation, migration, death, and other cell life-altering events. Detection of cell-secreted molecules is accomplished by sampling the collected waste fluid from each reservoir using immunoassay or optical analysis.

Integrated Measurement System:

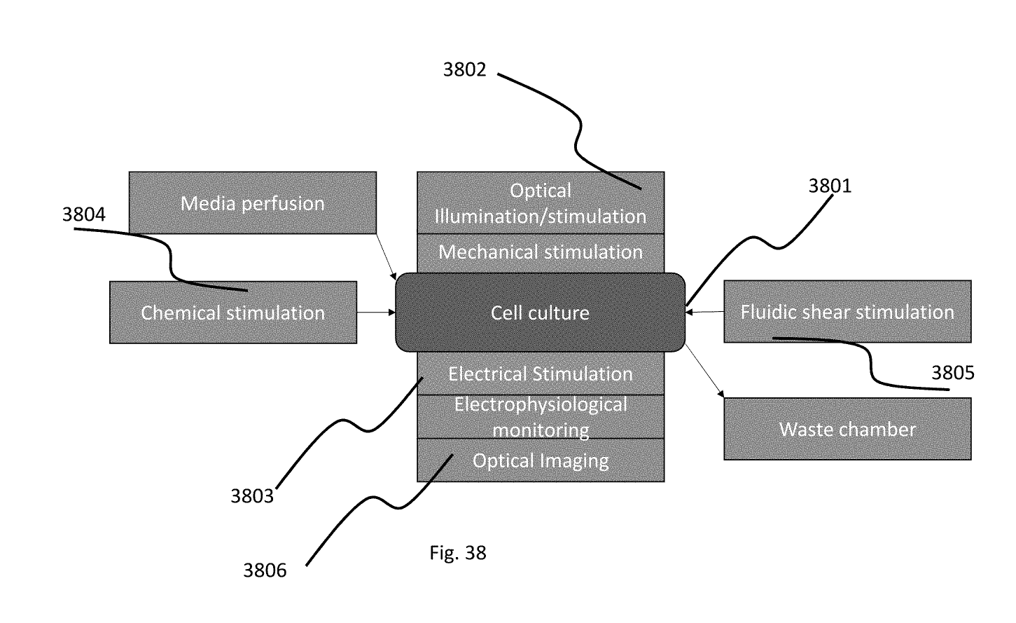

FIG. 38 shows the stimulation 3802, 3803, 3804, 3805 and recording of multiple parameter 3806 and fluidics of the system.

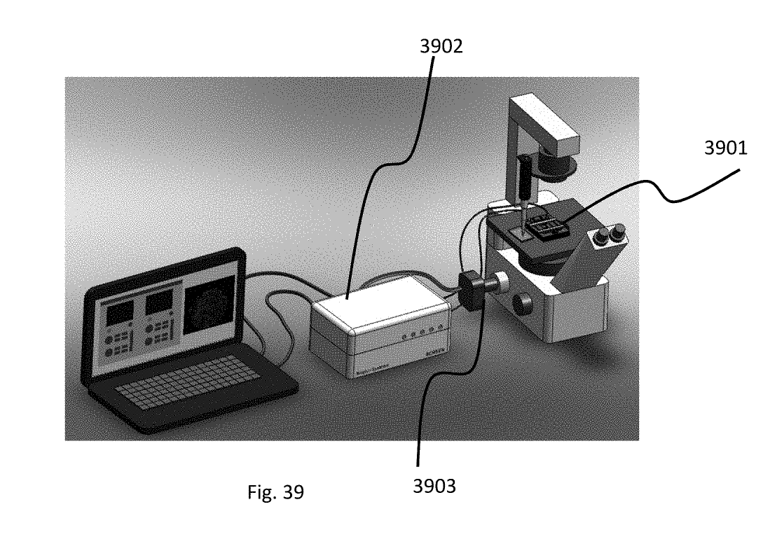

A standard imaging system with microscope 3901, camera 3903, control unit 3902 and xyz stage is used for the recording of the cells as shown in FIG. 39.

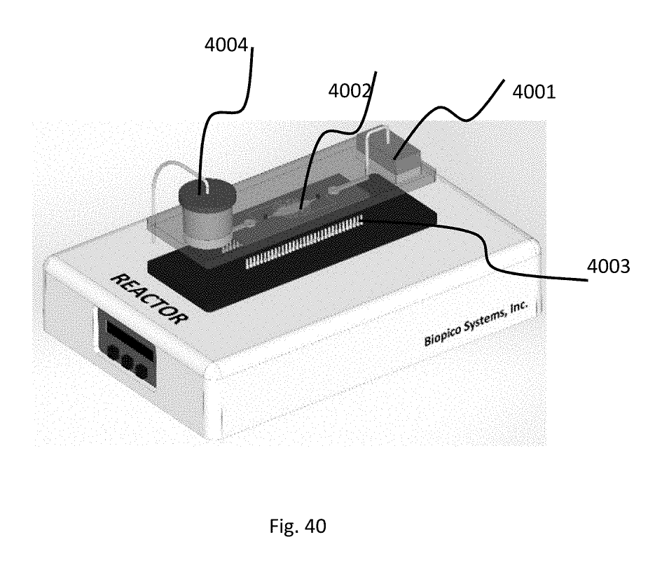

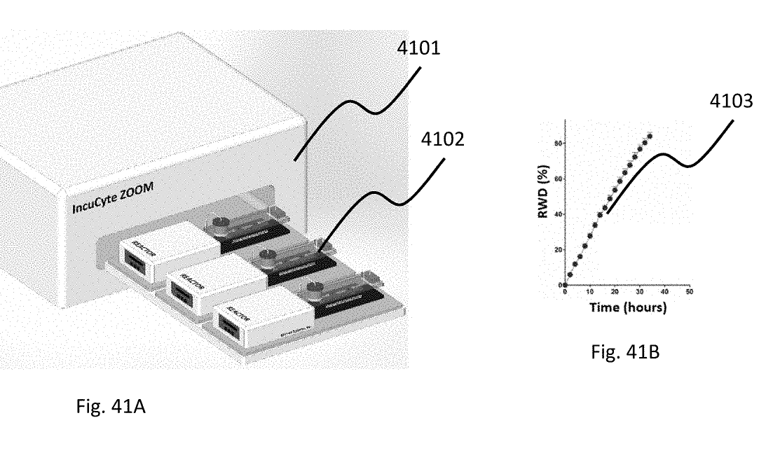

The integrated system with fluidic chip 4002, reservoir for reagents 4004, waste chamber 4001 and electrical recording 4003 is shown in FIG. 40.

The fluidic system 4102 is attached in commercial imaging system such as IncuCyte Zoom 4101 (FIG. 41A) for the real time imaging of cells and to develop a temporal performance 4103 of the cells as in FIG. 41B. However, the system is further developed to perform real time imaging and electrophysiological monitoring under stimulation.

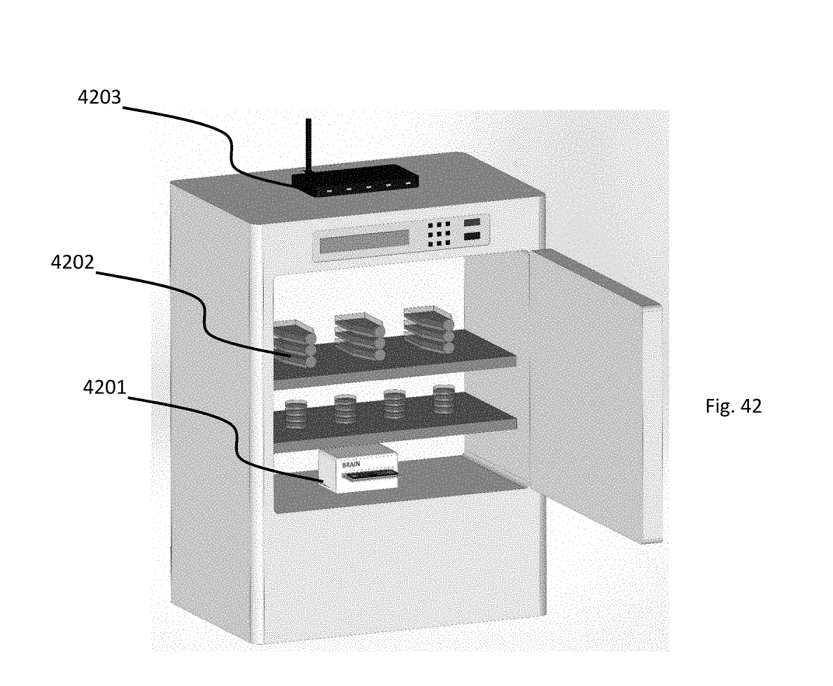

FIG. 42 shows the system 4201 with other cell culture flasks/devices in a general purpose incubator as an independent system. The electrophysiological signals are transmitted through a router 4203.

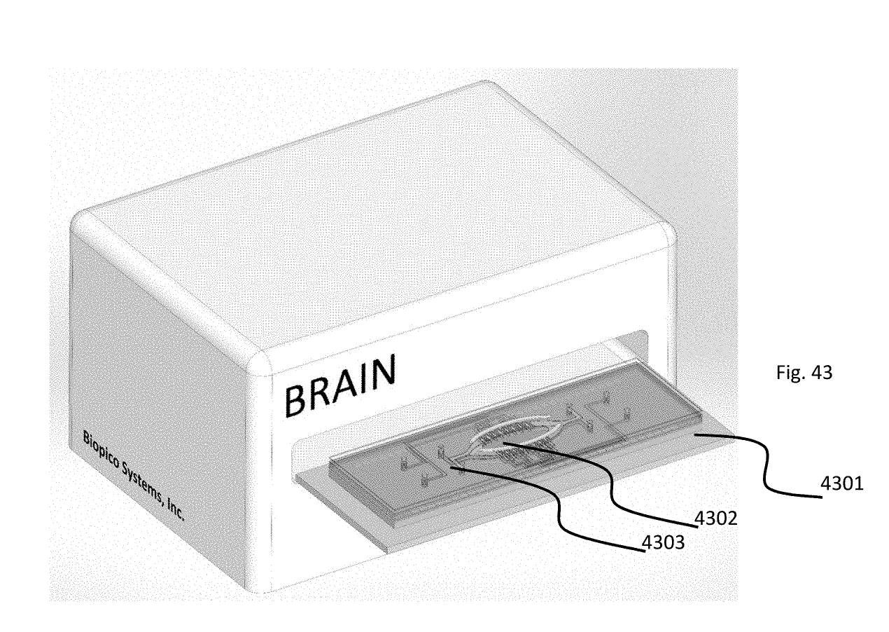

The system will have a user-friendly operation for loading cells and fluids using a trau 4301 as in FIG. 43. The fluidic ports 4303 are aligned to fluidic manifold and imaging of cells from the chip 4302 are carried out.

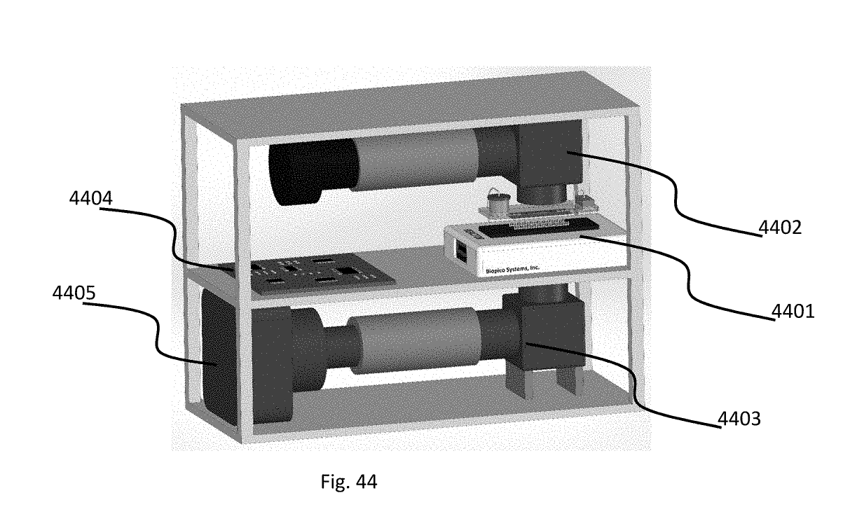

A portable inverted microscopic system with light illumination 4402 and objective 4403 (FIG. 44) is integrated to record optical images of the cells under investigation using a camera 4405.

### Design and Development of Assay

Drug Screening:

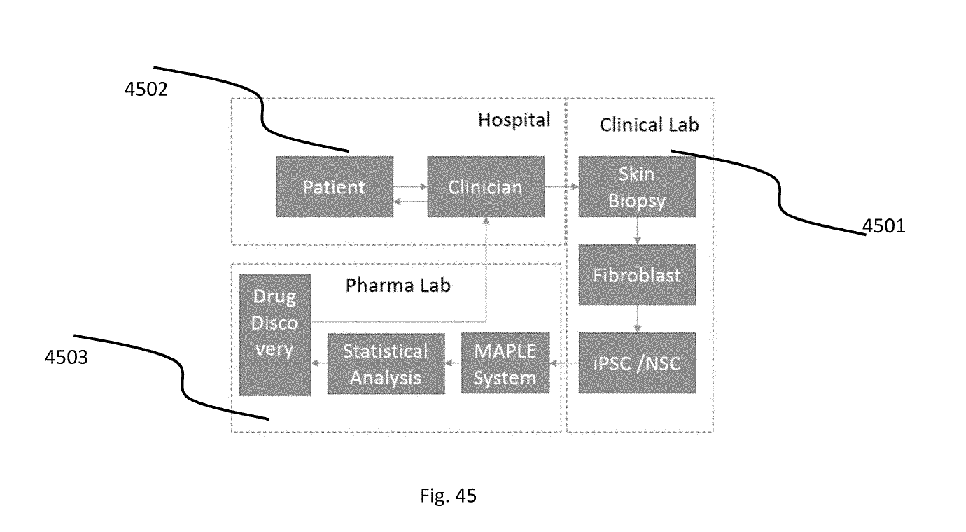

The hNSCs differentiated from induced pluripotent stem (iPS) cells of patientis with neurological disorders such as seizure and Fragile X, are developed and expanded. The NSCs will be loaded into the microfluidic reactors will be maintained with a constant perfusion of media. The effect of the drug dosage on control and diseased cell lines is studied. After culturing the cells for 3 weeks, the cells become more electrogenic and assessed electrophysiologically. The characteristics for Spike peak/valley heights and widths and spike/burst rates are correlated with the disease state of the cells. Such drug screening can be extended to personalized medicine using iPS cells from patients 4502 as shown in FIG. 45. Skin biopsy 4501 from patients undergoing treatment will be taken and iPS cells are formed which are converted to NSCs. Pharmacology experiments 4503 with the neural cells using electrophysiological signals will provide patient specific signals to clinicians.

Toxicity Screening:

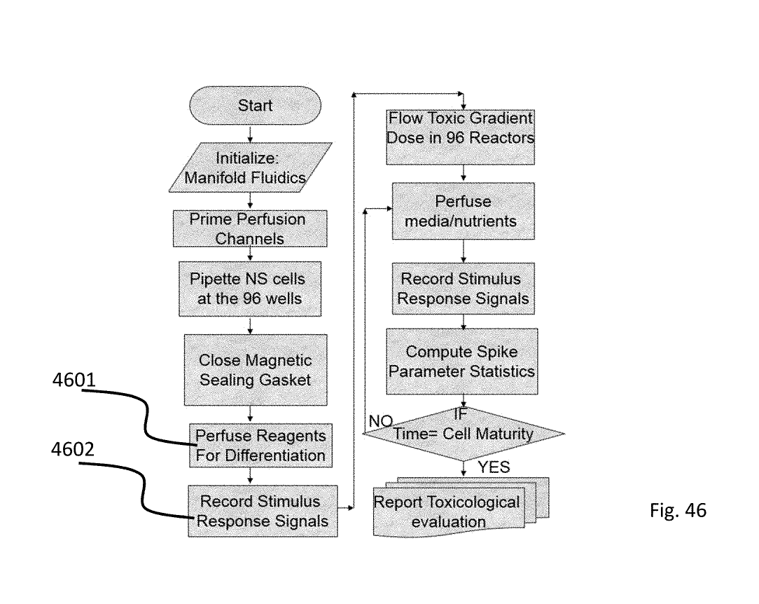

High-throughput chip is used for studying electrophysiological monitoring of NSC differentiation under toxins using a stimulation/recording protocol for field potential measurement experiments and data acquisition and data analysis. The system monitor the cells as they differentiate into neurons using spike characteristics and compare the spike/burst characteristics across different dosages of toxins for a given differentiated state of the neural cells as presented in FIG. 46. Toxin reagents are perfused for differentiation 4601 and corresponding electrophysiological signals 4602 are recorded.

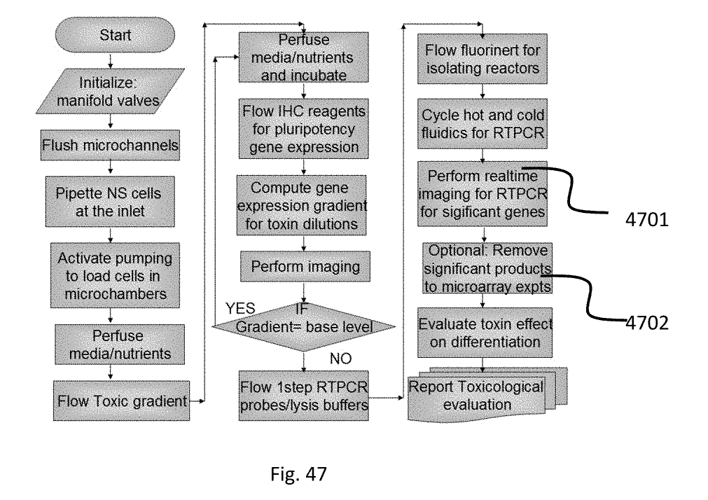

Cells after the electrophysiological monitoring assay can be retrieved 4702 from the reactors and analyzed for gene expression assay as in FIG. 47. Real Time PCR (RTPCR) can also be performed insitu on chip 4701.

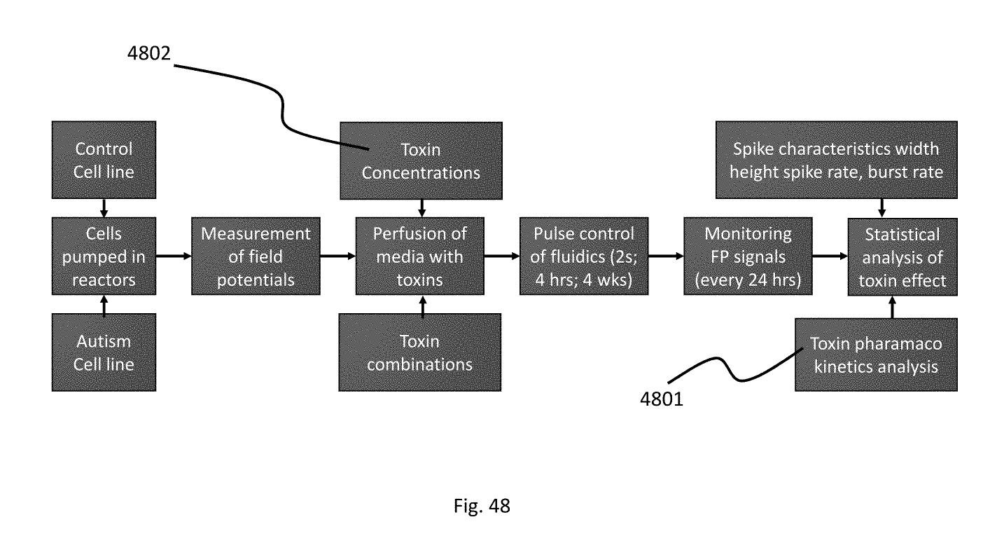

A protocol for toxicological assay 4801 to compare with control cell line and disease cell line using toxin concentration 4802 is shown in FIG. 48.

Transfection:

Cultured neurons are transfected with DNA in microfluidic environments using calcium phosphate transfection method. The system would retain the ease of use while improving transfection efficiencies, thus broadening its application in functional genetic analyses.

Organ on a Chip:

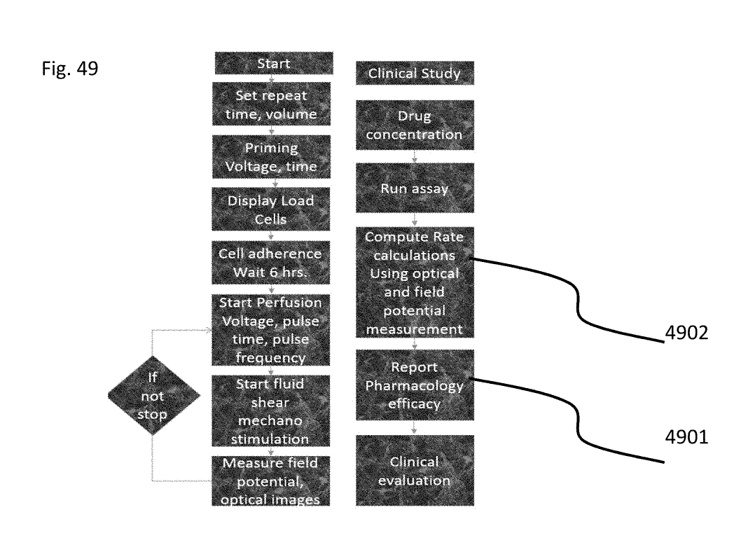

Using Cardiomyocytes mechanically stimulated and recorded optically and electrically, heart-on-a-chip is developed. An operational flow chart to perform heart-on-a-chip pharmacological assay 4901 using field potential signals and optical signals 4902 is shown in FIG. 49. Using brain endothelial cells and vascular cell constructed on two compartment with proteins such as claudin, occluding, and junction adhesion molecules and cytoplasmic accessory proteins tight Junction are created. The two compartment models are trying to demonstrate the barrier (composed of cells) and the fluid (blood) which undergoes shear stress. The system is able to fit in the foot print of the 96-well plate with several copies of the organ or multiple organs to perform drug screening. The cells will be plated on the chips and imaged with a time lapse study. After the cells are attached to the chip, may take 48-72 hours with media changes automated with the pump for periodic delivery of fluids. The images and electrophysiological measurements of the cells is performed periodically. The cells are maintained with constant stimulation, such as mechanical by recycling shear stress before or after the measurements. We study the effect of the drug dosage on control and stimulated cells. The characteristics for spikes and reaction rates are correlated with the drug concentration to establish the efficacy of a drug.

Disease modeling: Cells with diseases will be used in the reactors for drug screening studies and are compared with normal cells. Induced pluripotent stem cell technology has provided possibilities to model human disease in the culture dish. Reprogramming somatic cells from patients by differentiation into disease-relevant cell types can generate an unlimited source of human tissue.

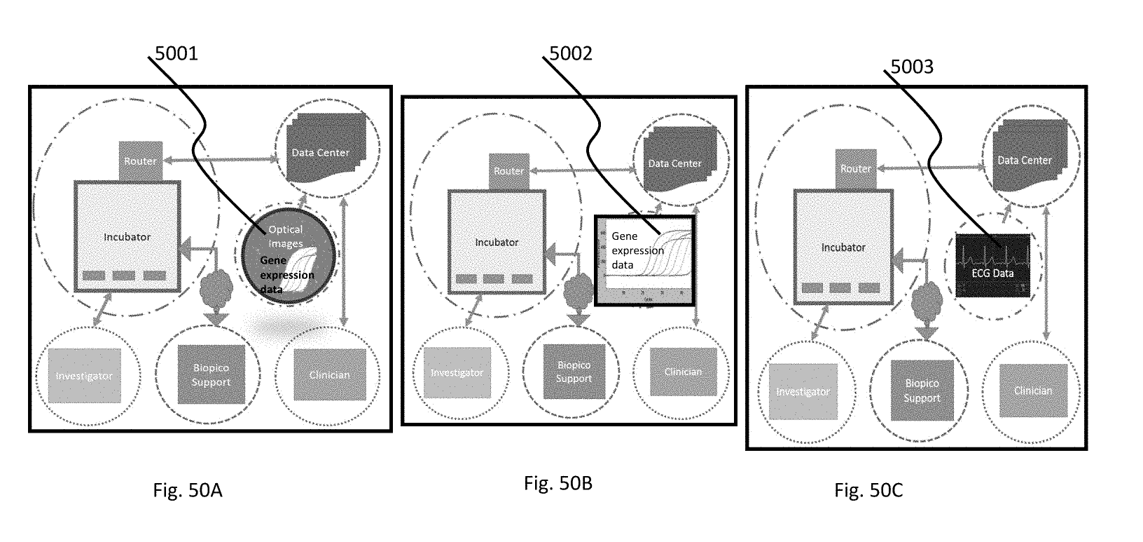

Integration of Human on a chip: Multiple organs are developed on high throughout chip using the cell lines from corresponding organs are direct differentiation using iPS cells. Communication between organs are achieved by microfluidic channels are proteins or molecules responsible for the cell signaling. Bioreactors for each organ that form semi-human or human on a chip can provide valuable information for drug discovery. The data from such studies can be archived in a server accessible to investigator, clinicians and support specialist as shown in FIG. 50A to FIG. 50C. We have developed methods for data communication across bioreactor experimental system on demand from a remote terminal and integration of multimodal measurements such as optical images 5001, gene expression data 5002 and patient's invivo measurements such as ECG 5003 or ECoG or optogenetic signals to transmit between investigators and clinicians through Biopico Systems' support team.

### EXAMPLES

### Example 1: Stem Cells in Chemical Toxicant Reactors for the Electrophysiological Evaluation of Neuronal Differentiation

Electrophysiological screening of stem cell differentiation in high-throughput invitro assays for thousands of chemicals provides a paradigm shift in predicting toxicological response in humans and to prioritize compounds for more extensive toxicological evaluation. In understanding the toxicity of such environmental chemical landscape, it is important to consider how chemicals affect embryos and fetuses, which are usually the most sensitive stages of the human life cycle. The advent of patient-derived induced pluoripotent stem cells (iPSC) provides a unique opportunity to explore such assessment of the effects of environmental chemicals on human prenatal development as they differentiate into any type of cell. Electrophysiological recording techniques have implicated in a diverse range of neurological disorders and cardiovascular systems. Fully differentiated neurons should have at least two types of voltage-gated ion channels (Nab, Kb) to generate a regenerative spike. Therefore, electrophysiological probing of patient iPSC-derived neurons can recapitulate the neuronal pathophysiology and respond to toxin or drug treatment. Fetuses, particularly males, are sensitive to multiple toxins such as environmental Bisphenol-A, lead, mercury, medications and a wide variety of other synthetic molecules, like pesticides. Exposure to these toxins during critical stages of development is thought to explain a large portion of congenital reproductive malformations. Toxic induced risk assessments are traditionally conducted on single chemicals and it is difficult to extrapolate results from a series of tests on individual chemicals to the effect of exposure to a complex mixture. Compared to rodent models for neurological disorders, patient-specific iPSC-derived neurons are expected to mimic disease pathophysiology more accurately and could be more easily adapted to high-throughput drug screen platforms. Noninvasive, monitoring of the effects of chemicals on differentiation or on differentiated cell directly in toxicological assays at various endpoints, including pluripotency, proliferation, apoptosis, survival and morphology could provide valuable information. However, presently, these tests are slow, costly, and provide only a limited estimation of human response to chemicals for such in vitro “disease in a dish” models. “Stem cells in Chemical Toxicant Reactors for the Electrophysiological Evaluation of Neuronal differentiation” provides high-throughput and reliable screening of toxicants using neural stem cells (NSC). The differentiation of multipotent NSCs in a 2-d culture format following the different dose patterns of the external toxicant stimuli is performed. In this device, the processes of liquid dilution, micro-scale cell culture, electrophysiological monitoring are integrated into a single device in a high-throughput format. This rapid screening system has the potential to provide species-specific toxicity information for diverse cellular responses of environmentally realistic exposures and to promote the understanding of chemical toxicity that disrupt the chemical balance and functioning of nerve cells. A few chemicals from the Tox21 library, is evaluated to assess the early exposure of which affects children's brain to formulate potentially preventable environmental causes of autism. High-throughput approaches to measure changes in electrophysiological markers after exposure to mixtures of toxicants has the potential to allow for the assessment of interactions such as additivity, synergism, or antagonism. The screening system will help in the understanding of the relationship of genetic sequence variability to human disease and sensitivity to chemical exposure to advance the individual health risk assessments findings from laboratory models to human risk. This study will contribute to the pursuit of developing of precise environmental causes of neurological disorders such as Autism Spectrum Disorder (ASD) and the development of new treatment options.

### Example 2: Lymphatic Cells in Yoked Microchannel for Pharmacological Study (LYMP) of Intestinal Diseases

Lymphatics responsible for transporting and maintaining fluids, lipids and immune cells may result in dysregulation of body fluid homeostasis, immune traffic impairment, and disturbances of lipid and protein reabsorption from the gut lumen. Therefore, understanding of the development, functions and the factors of lymphatics and their contribution to disease pathogenesis will help in our ability to accurately identify, categorize, treat, and prevent these diseases. There have been several attempts to construct in-vitro models that can recapitulate the biophysical environment seen in-vivo and to characterize their transport mechanisms including tissue-engineered models and transwell based models. But these models do not provide active control and quantitatively record the functional behavior of the lymphatic system to understand the modulation of lymphatic endothelial integrity. A fully automatic Lymphatic smooth muscle cells in Yoked Microchannel for Pharmacological study (LYMP) of intestinal diseases that integrates on-chip lymphatic model to study digestive system applications enables screening of extensive sets of experimental conditions within nanoliter volumes in a more controllable way and improve the ability to visualize, manipulate, and measure co-culture conditions of lymphatic vessel and the surrounding cells leading to greater understanding of the underlying causes of diseases. Our long-term goal is to provide robust, user-friendly, and cost effective culture platforms that can quantitatively screen and optimize drug candidates for lymphatic diseases with refined understanding of the molecular mechanisms behind the origin of these diseases. This LYMP system will shift current research paradigms through the development of novel cell-based tools in the understanding of disease pathways in drug discovery or optimization so that a true concord between biologists, clinicians, pharmaceutical companies and patients is achieved.

The Microfluidic LYMP System will set up an invitro model to mimic the interaction of lymphatic endothelium and intestinal smooth muscle cells for fluid entry to the lymphatic system. The smooth muscle cells in collecting lymphatics with series of lymphangions functional units allows for spontaneous contraction forming valves to facilitate the transport of lymph to the adjacent downstream lymphangion away from the tissues. The system can regulate tissue pressure and fluid status to study the uptake mechanisms of fluids into the lymphatic system. This system has the capability to incorporate drug stimuli gradient generator which will offer precise control over physiologic stresses, chemical signaling, and the degree of cell-cell interaction. If the stimuli in the fluid affects the Lymphatic endothelial cells (LEC), it relaxes smooth muscle cells and the vessel collapses resulting in fluid accumulation and edema in the spaces such as the microvilli. The diseases that can be studied using the LYMP system range from congenital malformations resulting in primary lymphangeictasias to dynamic processes of lymphatic growth, remodeling, and inflammation. Lymphatics play a role in Inflammatory bowel diseases (IBD) in gastrointestinal tract (e.g., Crohn's disease) or the colon (e.g., ulcerative colitis) and understanding the cause and consequences of lymphangitis is a key to the disease pathogenesis. Therefore, LYMP system has significant potential for evaluating pathological changes in lymphatic system associated with inflammatory diseases using multiple growth factors. The technique has significant potential for evaluating pathological changes in tissues associated with inflammatory diseases. The real-time electrophysiological measurements offer quantitative non-invasive monitoring of the effect of drug stimuli on the co-cultured LEC-intestinal smooth muscle (ISM) cells for days to weeks. This quantitative technique can provide the integrity of the tight junctions that govern solute transport across the paracellular space of the system. The lymphatic process from coordinated contractions of smooth muscle are derived from two basic patterns of electrical activity across the membranes of smooth muscle cells—slow waves and spike potentials. The smooth muscle cells maintain an electrical potential difference across their membranes with spontaneous fluctuation resulting slow waves of partial depolarization occurring 10 to 20 times per minute. Spiked action potentials that elicit muscle contraction and occur at the crests of slow waves are resulted by exposure of neurotransmitters released in their vicinity by enteric neurons. The neurotransmitters are released in response to drugs responsible for the pathological changes of IBD are monitored for the pharmacological study of intestinal diseases.

### Example 3: Microelectrophysiological Assessment of Pharmacology Using Labchip Electroencephalogram (MAPLE) for Neurological Diseases

Seizure disorders comprise the major symptoms for a whole host of neurological diseases and injuries. Finding the appropriate drug regimen to treat these disorders is arduous and time-consuming. Moreover, the appropriate drug regimen varies from patient to patient. Clinicians generally prescribe one medication and evaluate its effectiveness over weeks or months; each new drug or drug combination is similarly evaluated. At present, there are about 30 different medications that could be prescribed. Thus, there can be an arduous journey of many months before a proper drug cocktail for a given patient can be devised. Because of iPSC technology, there is now a tremendous opportunity to design a system that can evaluate the drug combinations and dosages in a timely manner and with greatly reduced patient risk. Importantly, during the clinical evaluation period mentioned above, the patient is being exposed to a number of different drugs with a number of different side effects; it is trial and error and the patient is the guinea pig. The electrophysiological functional assay using the MAPLE system could be used as a surrogate, allowing the evaluation of different pharmacological pathways and dosages, protecting the patient from the usual drug odyssey. In other words, one could screen a patient's cells with a variety of drugs known to be useful for seizures, for example, to more quickly arrive at the best drug or combination of drugs for that particular patient's seizures. “Microelectrophysiological Assessment of Pharmacology using Labchip Electroencephalogram (MAPLE) for neurological diseases” provides a high-throughput and reliable screening for patient-specific drugs using patient-derived neural cells. The development of the MAPLE system will focus on the differentiation of multipotent NSCs into neurons in a 2-d culture format. Initially, we will develop the system using two disease cell populations with excitatory or inhibitory drugs and anti-seizure medications that will form the basis of establishing a clinical MAPLE platform for personalized medicine.

It is difficult to create an animal model of a neurological disease, such as epilepsy or fragile X that entirely recapitulates the human disease. Furthermore, in vivo experiments are, of course, not feasible with humans, so an in vitro representation of the in vivo human brain would go a long way toward bridging the gap between basic science and clinical application. Such an in vitro system not only could be used to screen for therapeutic drugs but could also be used to probe for mechanistic correlates. The current most significant in vitro representation of the in vivo human brain is the induced pluripotent stem cell (iPSC)-based system. In this system, patient-derived somatic cells, such as fibroblasts, are reprogrammed to a pluripotent state, and then expanded and differentiated down the neural lineage. Neural stem cells thus produced can then be reliably converted into more terminally differentiated neural cell types, including neurons and glia. Thus, from any given patient with any given genetic background, an in vitro representation of their neural cells can be made and tested, both mechanistically, by comparing diseased cells to normal ones, and pharmacologically, by testing the cells' responses to particular drugs. Electrophysiological evaluation of seizure related neurons in the assays is important since the seizures result from an imbalance in the electrical activity of neurons. The high-throughput multi-electrode array-based assay to monitor the electrophysiological properties of diseased and healthy neurons and their responses to potential therapeutic agents is highly significant in that it allows the establishment of an assay an assay for personalized drug selection. The cell-based assay is performed in a perfusion format fitted with microfluidic channels consuming microliter to nanoliters of reagents, having short diffusion paths for quick reaction and fast analysis and a highly paralleled operation, and versatile and precise controls for fluid transport, mixing and concentration manipulations.

### Example 4: Regenerative Electromechanical Aided Chemical Stimulation with Transducers for Opto-Electrophysiological Recordings (REACTOR) to Support Cardiac Pharmacology

Biomechanical, electrical and chemical stresses or stimuli play a vital role for normal cardiac development and are shown to activate signal transduction pathways and subsequently regulate cardiac gene expression, proliferation and cell-growth. The responsiveness on the cellular level influences the mechanical function on the tissue and organ level and the ability to modulate cell biochemical reactions would help in the development of functional drug screening applications. There is an urgent clinical need to engineer functionally viable regenerative tissues using stress parameters that mimic the native environment. Such model systems with externally applied forces will not only further our understanding of therapeutic approaches to cardiac regeneration but also would enable to develop a drug screening function assay for cardiac diseases. Therefore, Biopico Systems Inc proposes to develop “Regenerative Electromechanical Aided Chemical stimulation with Transducers for Opto-electrophysiological Recordings (REACTOR) to support cardiac pharmacology”. This REACTOR system is validated in a Good manufacturing practice/Good Laboratory Practice (GMP/CLP) regulated environment for pre-clinical and subsequent clinical adaptation. The REACTOR system will be established as inexpensive, easily manipulated, easily reproducible, physiologically representative of human disease, and ethically sound system. Such system will provide complementary features such as electro mechanico chemical stimulations capabilities and electrophysiological monitoring in a fully automated system. A cell on bioreactors is an adaptive mechanical structure that both receives and responds to biochemical, biomechanical, and bioelectrical signals. Further mechanical stimulation of cells results in cell-generated responses for a variety of cell processes including differentiation, proliferation, extracellular matrix production, alignment, migration, adhesion, signaling, and morphology. During cardiomyopathy, Tgf-β signaling is thought to activate resident cardiac fibroblasts, leading to excessive fibroblast proliferation, cardiac fibrosis, and stiffening of the heart through excessive deposition of extracellular matrix. The high-throughput multi-electrode array-based assay to monitor electrophysiological properties cardiac cells and their responses to potential therapeutic agents is highly significant in that it allows the establishment of an assay for personalized drug selection. During continuous live-cell monitoring and analysis, cells are not disturbed by the observation and analysis and so repeated measures over time provide powerful insight into the time course of biology and provides greater control over critical assay conditions. The kinetic data enable novel powerful analyses such as rate measurements, time to threshold, and area under curve. Such cell-based assay in a perfusion format fitted with microfluidic channels will consume only microliter to nanoliters of reagents, avoid cell contamination, easily adapted to GMP/GLP and provide highly paralleled operation.

### Example 5: Blood-Brain-Barrier Reactors to Assay Invitro for Neurotherapeutics

The blood-brain barrier (BBB) is formed by the brain capillary endothelium and excludes large-molecule and more than 98% of all small-molecule drugs from the brain. There are few effective treatments for many central nervous system (CNS) disorders due to the minimal BBB transport of many potential CNS drugs. Our accelerated effort to develop invitro BBB model using the transport properties of molecular and cellular biology of the brain capillary endothelium could accelerate CNS drug delivery and drug discovery efforts in the molecular neurotherapeutics.

1: A method for high-throughput drug screening on cells on a multi-layer chip, the method comprising:
performing fluidic perfusion in at least one of a plurality of microfluidic reactors on a layer of the multi-layer chip;
loading a cell into the at least one of the plurality of microfluidic reactors;
stimulating the cell using at least one of an electrical pulse, a mechanical fluidic shear, a light, a sound, and a chemical fluidic pulse;
sensing one or more signals from the cells using one or more sensing electrodes in response to the stimulating.

2: The method of claim 1, wherein the loading comprises flowing the cells into the at least one of the plurality of microfluidic reactors, the plurality of microfluidic reactors arranged in a standard well-plate format connected by a microchannel that prevents contamination of a first microfluidic reactor of the plurality of microfluidic reactors from a second microfluidic reactor of the plurality of microfluidic reactors.

3: The method of claim 1, wherein performing fluidic perfusion comprises loading at least one of a drug, a toxin, and a reagent having a concentration gradient in the plurality of microfluidic reactors, the plurality of microfluidc reactors connected using one of a parallel configuration and a serial configuration, the serial configuratoin comprising an inlet spiral channel and an outlet spiral channel.

4: The method of claim 1, wherein the at least one of the plurality of microfluidic reactors comprises a reaction chamber, the reaction chamber coupled to a cell channel for the loading the cell and a plurality of perfusion channels for the fluidic perfusion.

5: The method of claim 1, wherein the plurality of microfluidic reactors are formed to co-culture two or more types of cells within a fluidic chip using two or more layers of fluidics separated by one or more filters.

6: The method of claim 5, wherein the plurality of microfluidic reactors are monitored within a co-culture system using one of impedance measurements and field potential measurement.

7: The method of claim 5, wherein the fluidic chip includes a plurality of channels wherein at least a first chanel connected to a second channel to exchange of one of a fluid, a plurality of molecules, and one or more cells between the first channel and the second channel, wherein at least one of the plurality of channels includes a plurality of cells adhered to an inside of the channel, the method further comprising:
forming a co-culture including two or more types of cells and one or more stimulants in at least one of the first channel and the second channel.

8: The method of claim 7, wherein the fluidic chip further comprises a first layer having spiral channels for lateral flow of cells or fluids, a filter layer under said first layer for separating the two or more types of cells, a first co-culture layer for stimulating a first type of cells using one or more chemicals, and a second co-culture layer for stimulating a second type of cells using one or more chemicals.

9: The method of claim 8, wherein the first co-culture layer is coupled to the second co-culture layer by connecting channels.

10: The method of claim 8, wherein one type of cells of the two or more types of cells responds to at least one of a chemical stimulant, an optical stimulant, a mechanical stimulant, and an electrical stimulant

11: The method of claim 1, wherein the mechanical fluidic shearing comprises pumping fluids using one or more piezo electric actuators (PZT) or PZT benders using alternating current (AC) voltages in a cascaded or perstastic mode of operation.

12: The method of claim 1, further comprising performing endothelial tight junction based cell assay using the multi-layer fluidic chip.

13: The method of claim 1, wherein the performing the fluidic perfusion comprises perfusing one of cell media and nutrient in the at least one of the plurality of microfluidic reactors.

14: The method of claim 1, further comprising:
delivering a drug into the cell using electroporation for pharmacological screening with one or more stimulii; and
monitoring the cell using one or more monitoring modalities.

15: The method of claim 1, further comprising remotely monitoring the cell using wirelessly transmitted data or wired network.

16: The method of claim 1, further comprising:
detecting the cells using a differential impedance measurement of one or more neighboring electrodes from the top, bottom, right or left in order to stimulate the cells or to measure field potential signals.

17: The method of claim 1, further comprising:
stimulating one or more cells using a stimulant across a plurality of concentric spiral micro/nano spaced interconnected channels including a plurality of inlets and a plurality of outlets, wherein the plurality of inlets and the plurality of outlets form tight junctions with the stimulant and the cells to transport fluids across a plurality of channels for drug screening application assessed by trans-epithelial electrical resistance measurements.

18: A multi-layer fluidic chip comprising:
a reaction layer having a plurality of microfluidic reactors, wherein fluidic perfusion is performed in at least one of the plurality of microfluidic reactors;
a microelectrode array layer under the reaction layer, the microelectrode array layer configured to:
stimulate at least one of a plurality of cells using at least one stimulating electrode; and
sense data from the at least one of the plurality of cells using at least one sensing electrode.

19: The multi-layer fluidic chip of claim 18, wherein the at least one of the plurality of microfluidic reactors comprises a reaction chamber coupled to a cell channel and a plurality of perfusion channels.

20: The multi-layer fluidic chip of claim 18, wherein the at least one of the plurality of cells is further stimulated using at least one of a chemical stimulant, a mechanical stimulation, and a fluidic shear stimulation in combination with an electrical stimulation from the stimulating electrode.
